(19) United States
(12) Patent Application Publication
(10) Pub. No.: US 2005/0026952 A1
(43) Pub. Date: Feb. 3, 2005

(54) Nicotinamide derivatives useful as PDE4 inhibitors

(75) Inventor: John Paul Mathias, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/895,873

(22) Filed: Jul. 20, 2004

Related U.S. Application Data
(60) Provisional application No. 60/497,128, filed on Aug. 22, 2003.

(30) Foreign Application Priority Data
Jul. 25, 2003 (GB) 0317482.8

(51) Int. Cl. A61K 31/4709; A61K 31/4439; C 07D 4 1/02

(52) U.S. Cl. 514/314; 514/337; 546/176; 546/277.4

(57) ABSTRACT

This invention relates to nicotinamide derivatives of general formula (I): 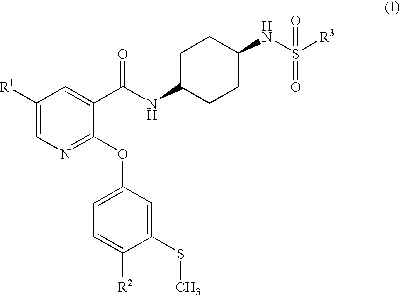
in which R¹, R² and R³ have the meanings defined herein, and to processes for the preparation of, intermediates used in the preparation of, compositions containing and the uses of such derivatives.

This invention relates to nicotinamide derivatives of general formula (I): 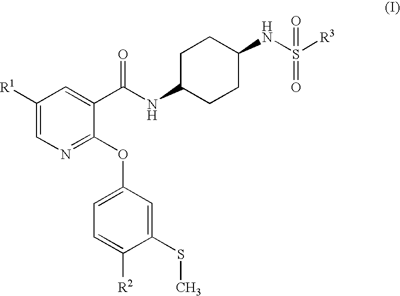
in which R¹, R² and R³ have the meanings indicated below, and to processes for the preparation of, intermediates used in the preparation of, compositions containing and the uses of such derivatives.

The 3′,5′-cyclic nucleotide phosphodiesterases (PDEs) comprise a large class of enzymes divided into at least eleven different families which are structurally, biochemically and pharmacologically distinct from one another. The enzymes within each family are commonly referred to as isoenzymes, or isozymes. A total of more than fifteen gene products is included within this class, and further diversity results from differential splicing and post-translational processing of those gene products. The present invention is primarily concerned with the four gene products of the fourth family of PDEs, i.e., PDE4A, PDE4B, PDE4C, and PDE4D. These enzymes are collectively referred to as being isoforms or subtypes of the PDE4 isozyme family.

The PDE4s are characterized by selective, high affinity hydrolytic degradation of the second messenger cyclic nucleotide, adenosine 3′,5′-cyclic monophosphate (cAMP), and by sensitivity to inhibition by rolipram. A number of selective inhibitors of the PDE4s have been discovered in recent years, and beneficial pharmacological effects resulting from that inhibition have been shown in a variety of disease models (see, e.g., Torphy et al., Environ. Health Perspect., 1994, 102 Suppl. 10, p. 79-84; Duplantier et al., J. Med. Chem., 1996, 39, p. 120-125; Schneider et al., Pharmacol. Biochem. Behav., 1995, 50, p. 211-217; Banner and Page, Br. J. Pharmacol., 1995, 114, p. 93-98; Barnette et al., J. Pharmacol. Exp. Ther., 1995, 273, p. 674-679 ; Wright et al., Can. J. Physiol. Pharmacol., 1997, 75, p. 1001-1008; Manabe et al., Eur. J. Pharmacol., 1997, 332, p. 97-107 and Ukita et al., J. Med. Chem., 1999, 42, p. 1088-1099). Accordingly, there continues to be considerable interest in the art with regard to the discovery of further selective inhibitors of PDE4s.

Successful results have already been obtained in the art with the discovery and development of selective PDE4 inhibitors. In vivo, PDE4 inhibitors reduce the influx of eosinophils to the lungs of allergen-challenged animals while also reducing the bronchoconstriction and elevated bronchial responsiveness occurring after allergen challenge. PDE4 inhibitors also suppress the activity of immune cells (including CD4⁺ T-lymphocytes, monocytes, mast cells, and basophils), reduce pulmonary edema, inhibit excitatory nonadrenergic noncholinergic neurotransmission (eNANC), potentiate inhibitory nonadrenergic noncholinergic neurotransmission (iNANC), reduce airway smooth muscle mitogenesis, and induce bronchodilation. PDE4 inhibitors also suppress the activity of a number of inflammatory cells associated with the pathophysiology of COPD, including monocytes/macrophages, CD4⁺ T-lymphocytes, eosinophils and neutrophils. PDE4 inhibitors also reduce vascular smooth muscle mitogenesis and potentially interfere with the ability of airway epithelial cells to generate pro-inflammatory mediators. Through the release of neutral proteases and acid hydrolases from their granules, and the generation of reactive oxygen species, neutrophils contribute to the tissue destruction associated with chronic inflammation, and are further implicated in the pathology of conditions such as emphysema. Therefore, PDE4 inhibitors are particularly useful for the treatment of a great number of inflammatory, respiratory and allergic diseases, disorders or conditions and for wounds and some of them are in clinical development mainly for treatment of asthma, COPD, bronchitis and emphysema.

The effects of PDE4 inhibitors on various inflammatory cell responses can be used as a basis for profiling and selecting inhibitors for further study. These effects include elevation of cAMP and inhibition of superoxide production, degranulation, chemotaxis, and tumor necrosis factor alpha (TNFa) release in eosinophils, neutrophils and monocytes.

Some nicotinamide derivatives having a PDE4 inhibitory activity have already been synthetized. For example, the patent application WO 98/45268 discloses nicotinamide derivatives having activity as selective inhibitors of PDE4D isozyme. The patent applications WO 01/57036 and WO 03/068235 also disclose nicotinamide derivatives which are PDE4 inhibitors useful in the treatment of various inflammatory allergic and respiratory diseases and conditions. However, there is still a huge need for additional PDE4 inhibitors that are good drug candidates. In particular, preferred compounds should bind potently to the PDE4 enzyme whilst showing little affinity for other receptors and enzymes. They should also possess favourable pharmacokinetic and metabolic activities, be non-toxic and demonstrate few side effects. Furthermore, it is also desirable that the ideal drug candidate will exist in a physical form that is stable and easily formulated.

The present invention therefore provides new nicotinamide derivatives of formula 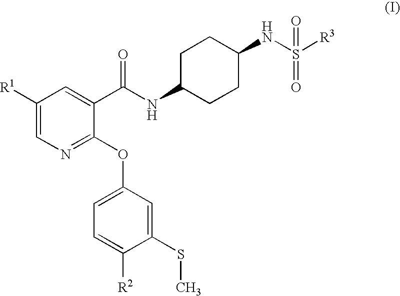
and pharmaceutically acceptable salts, solvates, polymorphs, and pro-drugs thereof wherein:

R¹ and R² are each independently selected from the group consisting of hydrogen, halo, and (C₁-C₃)alkyl;

R³ is an heteroaryl selected from: a 9- or 10-membered bicyclic heteroaryl containing from 1 to 3 heteroatoms selected from nitrogen, oxygen and sulphur and mixtures thereof; a phenyl ring; or a 5- or 6-membered heteroaryl containing from 1 to 3 nitrogen atoms; wherein heteroaryls of R³ are each independently optionally substituted by one or more groups selected from OH, CN, halo, (C₁-C₄)alkyl, hydroxy(C₁-C₄)alkyl, halo(C₁-C₄)alkyl, (C₁-C₄)alkoxy and hydroxy(C₂-C₄)alkoxy.

Preferably R¹ is H, F, Cl or methyl, more preferably R¹ is F.

Preferably R² is H or F, more preferably R² is H.

When R³ is a bicyclic heteroaryl then preferably said heteroaryl is a C-linked 9- or 10-membered bicyclic heteroaryl containing from 1 to 3 heteroatoms selected from nitrogen, oxygen and sulphur and mixtures thereof.

When R³ is a C-linked 9- or 10-membered bicyclic heteroaryl, more preferably R³ is selected from the group consisting of: indole, isoindole, indolizine, indazole, benzoimidazole, imidazopyridine, pyrrolopyridazine, pyrrolopyridine, benzotriazole, pyrazolopyridine, imidazopyridine, quinoline, isoquinoline, cinnoline, quinoxaline, quinazoline, phthalazine, naphthyridine, benzofuran, benzothiophene, benzoxazole, benzothiazole, benzothiadiazole and benzoxadiazole.

When R³ is a C-linked 9- or 10-membered bicyclic heteroaryl, especially preferred R³ groups are selected from the group consisting of: quinoline, 2,1,3-benzothiadiazole and 2,1,3-benzoxadiazole.

Preferred optional substituent groups for the C-linked 9- or 10-membered bicyclic heteroaryl of R³ are selected from OH, methyl, ethyl, hydroxymethyl, hydroxyethyl, hydroxyethoxy, F and Cl. Especially preferred as an optional substitutent group for the C-linked 9- or 10-membered bicyclic heteroaryl of R³ is OH.

When R³ is a phenyl group then preferably said phenyl group is optionally substituted by one or more substituents selected from OH, methyl, ethyl, hydroxymethyl, hydroxyethyl, hydroxyethoxy, F and Cl.

When R³ is a phenyl group then more preferably said phenyl group is optionally substituted by one or more substituents selected from OH, methyl, F, CN and CF₃.

When R³ is a 5- or 6-membered heteroaryl then preferably R³ is an optionally substituted C-linked 5-membered heteroaryl containing 2 or 3 nitrogen atoms.

When R³ is a 5 or 6 membered heteroaryl compound then more preferably R³ is selected from pyrazole and imidazole.

When R³ is a 5- or 6-membered heteroaryl then highly preferred are C-linked 5-membered heteroaryls containing 2 nitrogen atoms optionally substituted by one or more groups selected from (C₁-C₃)alkyl and (C₁-C₃)alkoxy.

When R³ is a 5- or 6-membered heteroaryl then especially preferred are C-linked 5-membered heteroaryls containing 2 nitrogen atoms optionally substituted by one or more methyl groups.

Preferred compounds according to the present invention are selected from the group consisting of:
Syn-N-(4-Benzenesulfonylamino-cyclohexyl)-5-fluoro-2-(3-methylsulfanyl-phenoxy)-nicotinamide;
Syn-5-Fluoro-N-[4-(3-fluoro-benzenesulfonylamino)-cyclohexyl]-2-(3-methylsulfanyl-phenoxy)-nicotinamide;
Syn-5-Fluoro-N-[4-(4-fluoro-benzenesulfonylamino)-cyclohexyl]-2-(3-methylsulfanyl-phenoxy)-nicotinamide;
Syn-5-Fluoro-2-(3-methylsulfanyl-phenoxy)-N-[4-(toluene-3-sulfonylamino)-cyclohexyl]-nicotinamide;
Syn-5-Fluoro-2-(4-methylsulfanyl-phenoxy)-N-[4-(toluene-3-sulfonylamino)-cyclohexyl]-nicotinamide;
Syn-N-[4-(3-Cyano-benzenesulfonylamino)-cyclohexyl]-5-fluoro-2-(3-methylsulfanyl-phenoxy)-nicotinamide;
Syn-5-Fluoro-2-(3-methylsulfanyl-phenoxy)-N-[4-(2-trifluoromethyl-benzenesulfonylamino)-cyclohexyl]-nicotinamide;
Syn-5-Fluoro-N-[4-(1-methyl-1H-imidazole-4-sulfonylamino)-cyclohexyl]-2-(3-methylsulfanyl-phenoxy)-nicotinamide;
Syn-N-[4-(3,5-Dimethyl-1H-pyrazole-4-sulfonylamino)-cyclohexyl]-5-fluoro-2-(3-methylsulfanyl-phenoxy)-nicotinamide;
Syn-5-Fluoro-2-(3-methylsulfanyl-phenoxy)-N-[4-(quinoline-8-sulfonylamino)-cyclohexyl]-nicotinamide;
Syn-N-[4-(Benzo[1,2,5]oxadiazole-4-sulfonylamino)-cyclohexyl]-5-fluoro-2-(3-methylsulfanyl-phenoxy)-nicotinamide;
Syn-N-[4-(Benzo[1,2,5]thiadiazole4-sulfonylamino)-cyclohexyl]-5-fluoro-2-(3-methylsulfanyl-phenoxy)-nicotinamide;
Syn-N-[5-Hydroxy-4-(benzo[1,2,5]thiadiazole4-sulfonylamino)-cyclohexyl]-5-fluoro-2-(3-methylsulfanyl-phenoxy)-nicotinamide;
Syn-5-Fluoro-N-[4-(2-hydroxy-4-methyl-benzenesulfonylamino)-cyclohexyl]-2-(3-methylsulfanyl-phenoxy)-nicotinamide; or
Syn-5-Fluoro-N-[4-(2-hydroxy-5-methyl-benzenesulfonylamino)-cyclohexyl]-2-(4-fluoro-3-methylsulfanyl-phenoxy)-nicotinamide
   and pharmaceutically acceptable salts, solvates, polymorphs and pro-drugs thereof.

Especially preferred compounds herein are selected from the group consisting of:
Syn-5-Fluoro-2-(3-methylsulfanyl-phenoxy)-N-[4-(quinoline-8-sulfonylamino)-cyclohexyl]-nicotinamide;
Syn-N-[4-(Benzo[1,2,5]thiadiazole4-sulfonylamino)-cyclohexyl]-5-fluoro-2-(3-methylsulfanyl-phenoxy)-nicotinamide; and
Syn-5-Fluoro-N-[4-(2-hydroxy4-methyl-benzenesulfonylamino)-cyclohexyl]-2-(3-methylsulfanyl-phenoxy)-nicotinamide
   and pharmaceutically acceptable salts, solvates, polymorphs and prodrugs thereof.

The present invention additionally provides compounds of formula (I) wherein R¹, R², and R³ are as previously defined and wherein the optional substituent groups of R³ additionally comprise hydroxymethoxy.

It has been found that these nicotinamide derivatives are inhibitors of PDE4 isoenzymes, particularly useful for the treatment of inflammatory, respiratory and allergic diseases and conditions or for wounds.

In the here above general formula (I), halo denotes a halogen atom selected from the group consisting of fluoro, chloro, bromo and iodo in particular fluoro or chloro.

(C₁-C₃)alkyl or (C₁-C₄)alkyl radicals denote a straight-chain or branched group containing respectively 1 to 3 and 1 to 4 carbon atoms. This also applies if they carry substituents or occur as substituents of other radicals, for example in (C₁-C₄)alkoxy radicals, hydroxy(C₁-C₄)alkyl radicals, halo(C₁-C₄)alkyl radicals and hydroxy(C₂-C₄)alkoxy. Examples of suitable (C₁-C₃)alkyl and (C₁-C₄)alkyl radicals are methyl, ethyl, n-propyl, iso-propyl, n-butyl, iso-butyl, sec-butyl and tert-butyl. Examples of suitable (C₁-C₄)alkoxy and (C₂-C₄)alkoxy radicals are methoxy, ethoxy, n-propyloxy, iso-propyloxy, n-butyloxy, iso-butyloxy, sec-butyloxy and tert-butyloxy. Hydroxy(C₁-C₄)alkyl and hydroxy(C₂-C₄)alkoxy radicals may contain more than one hydroxy group (—OH). According to a preferred embodiment of said invention, such radicals contain one hydroxy substituent. Examples of suitable hydroxy(C₁-C₄)alkyl radicals are hydroxymethyl, 1-hydroxyethyl or 2-hydroxyethyl. Accordingly, halo(C₁-C₄)alkyl radicals may contain more than one halo group.

In the hereabove general formula (I), “5- or 6-membered heteroaryl” means a radical of a monocyclic aromatic system having 5 or 6 ring members, which contains 1, 2 or 3 nitrogen (N) atom(s) depending in number and quality of the total number of ring members. Examples of additional, optional heteroatoms are oxygen (O) and sulphur (S). If several heteroatoms are contained, these can be identical or different. Heterocyclic radicals can also be unsubstituted, monosubstituted or polysubstituted, as indicated in the definition of R³ hereabove for general formula (I) according to the present invention. Any suitable 5 or 6 membered heteroaryl containing from 1 to 3 nitrogen (N) atoms may be used. Preferred examples of suitable monocyclic heterocyclic radicals are the radicals containing 2 nitrogen atoms derivated from: pyrazole and imidazole. In the hereabove general formula (I), “9- or 10-membered bicyclic heteroaryl” means a radical of a bicyclic aromatic system having 9 or 10 ring members, which contains 1, 2 or 3 nitrogen (N) atom(s) depending in number and quality of the total number of ring members. Examples of additional, optional heteroatoms are oxygen (O) and sulphur (S). If several heteroatoms are contained, these can be identical or different. Said heteroaryl radicals can also be unsubstituted, monosubstituted or polysubstituted, as indicated in the definition of R³ hereabove for general formula (I) according to the present invention. Examples of suitable bicyclic heteroaryl radicals are the radicals derivated from indole, isoindole, indolizine, indazole, purine, napthyridine, phthalazine, quinoline, quinazoline, quinoxaline, cinnoline, isoquinoline, benzoimidazole, imidazo[1,2-a]pyridine, benzotriazole, pyrazolo[1,5-a]pyridine and pyrazolopyrimidine. Particularly preferred are the bicyclic heterocyclic radicals selected from indole, isoindole, indolizine, indazole, benzoimidazole, imidazopyridine, pyrrolopyridazine, pyrrolopyridine, benzotriazole, pyrazolopyridine, imidazopyridine, quinoline, isoquinoline, cinnoline, quinoxaline, quinazoline, phthalazine, naphthyridine, benzofuran, benzothiophene, benzoxazole, benzothiazole, benzothiadiazole and benzoxadiazole. More preferred are quinoline, 2,1,3-benzothiadiazole and 2,1,3-benzoxadiazole.

Nitrogen heteroaryl radicals can also be present as N-oxides or as quaternary salts.

In the general formula (I) according to the present invention, when a radical is mono- or poly-substituted, said substituent(s) can be located at any desired position(s). Also, when a radical is polysubstituted, said substituents can be identical or different, unless otherwise stated.

The nicotinamide derivatives of the formula (I) can be prepared using conventional procedures such as by the following illustrative methods in which R¹, R² and R³ are as previously defined for the nicotinamide derivatives of the formula (I) unless otherwise stated.

The compounds of formula (I) may be prepared by the methods disclosed hereunder, and exemplified in the Examples and Preparations. Other methods may be used in accordance with the skilled person's knowledge.

Unless otherwise provided herein:
PyBOP® means Benzotriazol-1-yloxytris(pyrrolidino)phosphonium hexafluorophosphate;
PyBrOP® means bromo-tris-pyrrolidino-phosphonium hexafluoro-phosphate;
CDI means N,N′-carbonyldiimidazole;
WSCDI means 1-(3-dimethylaminopropyl)-3-ethylcarbodiimide hydrochloride;
Mukaiyama's reagent means 2-chloro-1-methylpyridinium iodide;
HATU means O-(7-Azabenzotriazol-1-yl)-N,N,N′N′-tetramethyluronium hexafluorophosphate;
HBTU means O-Benzotriazol-1-yl-N,N,N′N′-tetramethyluronium hexafluorophosphate;
DCC means N,N′-dicyclohexylcarbodiimide;
CDI means N,N′-carbonyldiimidazole;
HOAT means 1-hydroxy-7-azabenzotriazole;
HOBT means 1-hydroxybenzotriazole hydrate;
Hünig's base means N-ethyldiisopropylamine;
Et₃N means triethylamine;
NMM means N-methylmorpholine;
NMP means 1-methyl-2-pyrrolidinone;
DMAP means 4-dimethylaminopyridine;
NMO means 4-methylmorpholine N-oxide;
KHMDS means potassium bis(trimethylsilyl)amide;
NaHMDS means sodium bis(trimethylsilyl)amide;
Boc means tert-butoxycarbonyl;
CBz means benzyloxycarbonyl;
MeOH means methanol, EtOH means ethanol, and EtOAc means ethyl acetate;
THF means tetrahydrofuran; DMSO means dimethyl sulphoxide; DCM means dichloromethane; DMF means N,N-dimethylformamide; ACOH means acetic acid; TFA means trifluoroacetic acid; RT means room temperature; 3° means tertiary; eq means equivalents; Me means methyl; Et means ethyl; Bn means benzyl; other abbreviations are used in accordance with standard synthetic chemistry practice.
   Route A

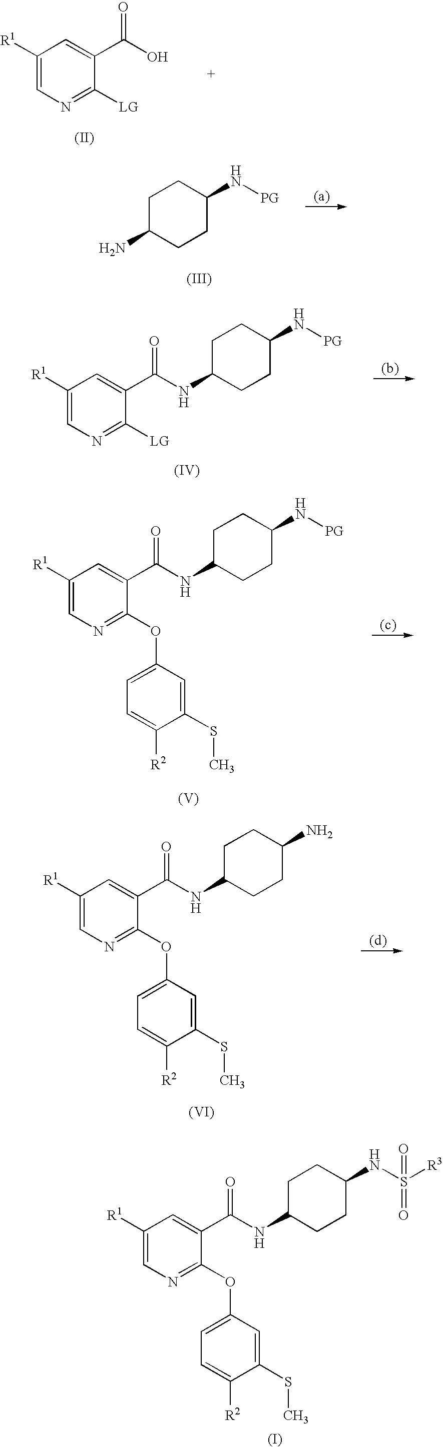

Nicotinic acids or acid derivatives of formula (II) are either available commercially or may be obtained by analogy with the methods of Haylor et. al. (EP 0634413 examples 9 and 10, pages 12-13), or Marzi et. al. (European Journal of Org. Chem. 2001 (7), 1371-1376). The protected amines of formula (III) are either available commercially or may be prepared by analogy with the method of Oku et al (WO 99/54284, for example, at page 80, preparation 77(1)).

In the Route A, R¹, R² and R³ are as previously defined, PG is a suitable amine protecting group, typically Boc, CBz or Bn, and preferably Boc, and LG is a suitable leaving group, typically halo, and preferably Cl.

Step (a)-Acid-amine Coupling

This acid/amine coupling may be undertaken by using either:
(i) an acyl chloride derivative of acid (II)+amine (III), with an excess of acid acceptor in a suitable solvent; or
(ii) the acid or acid derivative (II) with a conventional coupling agent +amine (III), optionally in the presence of a catalyst, with an excess of acid acceptor in a suitable solvent.

Typically the conditions are as follows:
(i) acid chloride of acid (II) (generated in-situ), an excess of amine (III), optionally with an excess of 3° amine such as Et₃N, Hünig's base or NMM, in DCM or THF, without heating for 1 to 24 hrs; or
(ii) acid (II), WSCDI/DCC/CDI optionally in the presences of HOBT or HOAT, an excess of amine (III), with an excess of NMM, Et₃N, Hünig's base in THF, DCM or EtOAc, at RT for 4 to 48 hrs; or, acid (II), PYBOP®/PyBrOP®/Mukaiyama's reagent/HATU/HBTU, an excess of amine (III), with an excess of NMM, Et₃N, Hünig's base in THF, DCM or EtOAc, at RT for 4 to 24 hrs.

The preferred conditions are: treatment of the acid chloride of (II) (generated in situ) about 1.1 equivalent of amine (III) and from about 2 to about 3 equivalents of Hünig's base or triethylamine in DCM at RT for about 18 hours, as illustrated by Preparation 6 herein.

Step (b)-Ether Formation

Substitution of the leaving group, LG, wherein said leaving group is for example a halogen and is preferably chlorine, of the compound (IV) with a substituted phenol to give compounds of formula (V).

Compounds of general formula (V) can be prepared from compounds of general formula (IV) via treatment with a suitable base, in a suitable solvent, in the presence of an excess of an optionally substituted, 3-methylsulphanyl-phenol. Alkali metal salts are used as the base (e.g. Cs₂CO₃, K₂CO₃, NaH) and MeCN, dioxan, or N,N-dimethylformamide are suitable solvents for use. The reaction is carried out at elevated temperature. Optionally the reaction may be carried out in the presence of a catalyst (eg imidazole, DMAP).

Preferred conditions are: reaction of compound (IV), wherein the LG is chlorine, with from about 1 to about 2.5 equivalents of an optionally substituted, 3-methylsulphanyl-phenol in the presence of an excess of caesium carbonate in dioxan at about the reflux temperature of the reaction, for from about 24 to about 72 hours. Exemplified herein by Preparation 12.

Step (c)-Removal of Protecting Group

Deprotection of the N protecting group (PG), from compounds of general formula (V) to provide compounds of general formula (VI) is undertaken using standard methodology, as described in “Protective Groups in Organic Synthesis” by T.W. Greene and P. Wutz.

For example when PG is Boc, the suitable conditions are: treatment of compound (V) with a strong acid (e.g. TFA, HCl), in a suitable solvent such as for example dioxan or DCM at room temperature. Preferred conditions herein for removal of a Boc group are treatment with hydrochloric acid in dioxan (eg 4M) and DCM at RT for about 3 hours or treatment with HCl saturated DCM for about 3 hours. Exemplified herein by preparations 13 and 14.

Step (d)-Formation of Sulphonamide

Compounds of the formula (I) may be prepared by a reaction of amine (VI) with a suitable compound of the formula R³SO₂LG, where LG is a suitable leaving group, typically halo and preferably Cl, by analogy with the methods previously described in Route A, step (a).

The preferred conditions are:
(i) treatment of amine (VI) with about 1.5 equivalents of R³SO₂Cl in the presence of excess Et₃N in dichloromethane at between RT and the reflux temperature of the reaction for from about 18 to about 48 hours; or
(ii) treatment of a mixture of amine (Vl) with WSCDI, HOBT, and about 1 equivalent of R³SO₂Cl, with an excess of 3° amine base (Hünig's base, Et₃N or NMM) in N,N-dimethylformamide, at RT for about 18 hours.

Examples 1 to 13 herein are illustrative of the conversion of compounds of formula (VI) to compounds of formula (I). Compounds of formula R³SO₂LG, are either commercially available, or may be obtained using standard methodology.

Route B

Compounds of general formula (I) may alternatively be prepared by Route B:

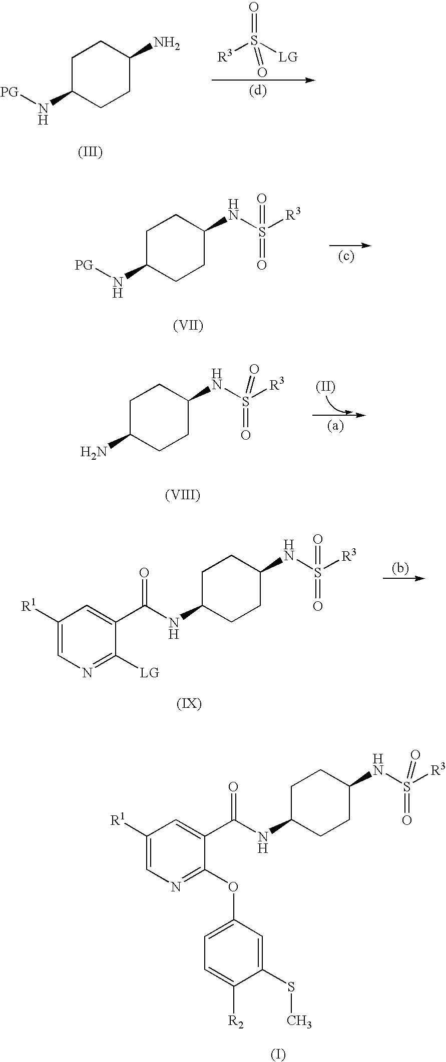

The compound of formula (VII) may be prepared from the amine (III) by reaction with R³SO₂LG according to the methods described previously in step (d), Route A.

The de-protected amine compound of general formula (VIII) may be prepared form the protected amine compound of general formula (VII) via removal of the protecting group PG, preferably a Boc group, by analogy to the methods described previously in step (c), Route A.

The amide compounds of general formula (IX) may be prepared by reaction of the amine of general formula (VIII) with the acid (II) according to the methods described previously in steps (a) and (d), Route A.

Compounds of formula (I) may be prepared by substitution of the leaving group, LG, of the compounds of formula (IX) by an optionally substituted, 3-methylsulphanyl-phenol group as described previously in step (b), Route A.

Route C

Compounds of general formula (I) may alternatively be prepared by Route C:

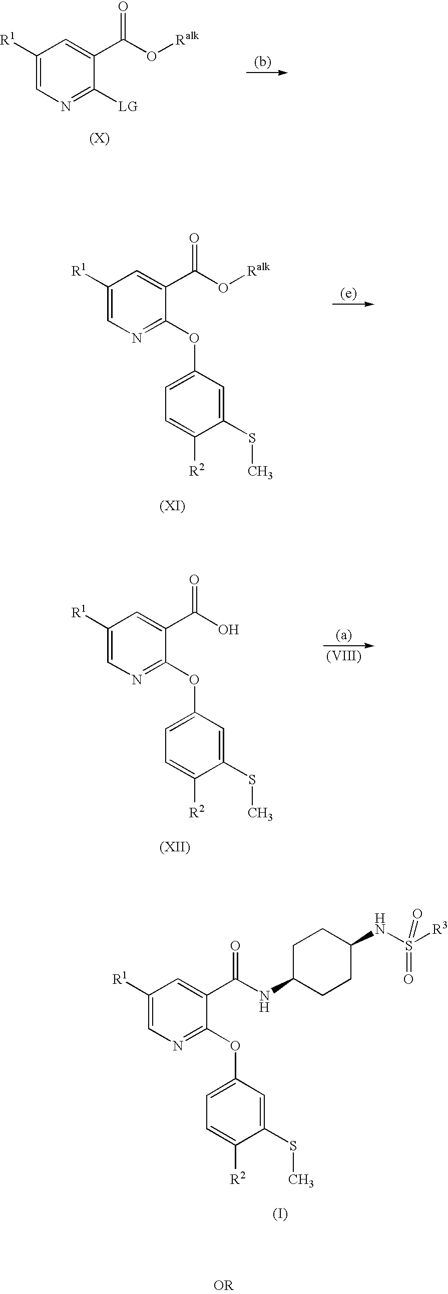

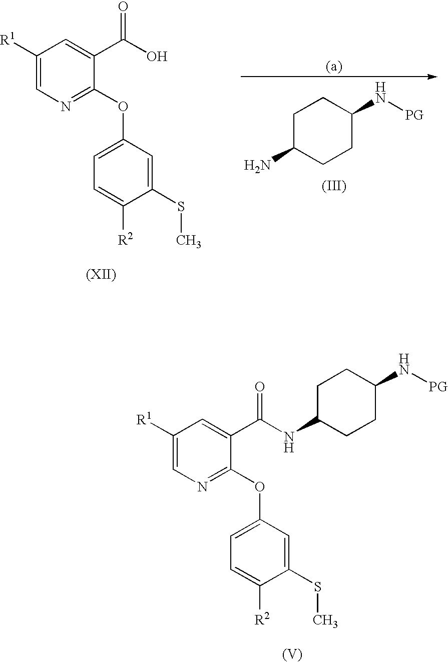

R^{alk} represents a C₁-C₄ alkyl group or Bz, preferably a C₁-C₃ alkyl group and more preferably Me or Et.

Compounds of formula (X) are either available commercially or may be obtained from the compounds of formula (II), using standard esterification conditions. The protected amines of formula (III) are either available commercially or may be prepared by analogy with the method of Oku et. al. (WO 99/54284) as described hereinbefore.

Compounds of formula (XI) may be prepared by reaction of the ester (X) with optionally substituted, 3-methylsulphanyl-phenol, as described previously in step (b), Route A. Suitable optional catalysts for use in this reaction include Cul. Preferred conditions for use herein are treatment with caesium carbonate in dioxan at about 100° C. for about 48 hours. Exemplified herein by Preparation 9.

Step (e)-Ester Hydrolysis

Hydrolysis of the ester (XI) may be achieved in the presence of acid or base, and in a suitable solvent, optionally at elevated temperature to afford the acid (XII). Typically, the ester (XI) is treated with a suitable base such as an alkali metal hydroxide (eg LiOH, NaOH, CsOH) in aqueous solvent (MeOH, EtOH, dioxan, THF) at between RT and the reflux temperature of the reaction, to give the acid (XII). Preferred conditions herein provide for treatment of ester (XI) in THF with about 2.5 equivalents of a 1M aqueous solution of LiOH in THF at RT for about 2 hours. Exemplified herein by Preparation 10.

Alternatively compounds of formula (IX) may be prepared from compounds of formula (II) by reaction with optionally substituted 3-methylsulphanyl-phenol, as described previously in step (b), Route A.

Reaction of the acid of formula (XII) with the amine of formula (VIII) as described previously in Route A, step (a) and Route B, step (a) provides the compounds of formula (I).

Alternatively where acid (XII) is reacted with a protected amine of formula (III), in accordance with the methods detailed in Route A, step (a), then protected amine of formula (V) is produced. Exemplified herein by preparation 11.

Such protected amine compounds of formula (V) can be de-protected by the methods described in Route A, step (c) and subsequently reacted with a suitable acid of formula R³COOH as described in Route A, step (d) to provide compounds of formula (I).

Certain R³ groups may undergo further functional group interconversions and transformations, such as demethylation of a methoxy group by treatment with HBr in acetic acid, or by treatment with BBr₃ in dichloromethane (as illustrated by Examples 14 to 16 hereinafter).

For example, protected compounds of the formula PGR³SO₂LG (such as the compound of Preparation 15) may equally be employed in the transformations described for Route A, step (d) to provide a protected sulphonamide (such as the compounds of Preparations 16, 17 and 18 herein) and followed by a suitable de-protection step as detailed hereinafter to furnish compounds of formula (I) (as illustrated by Examples 14 to 16 herein).

All of the above reactions and the preparations of novel starting materials using in the preceding methods are conventional and appropriate reagents and reaction conditions for their performance or preparation as well as procedures for isolating the desired products will be well-known to those skilled in the art with reference to literature precedents and the examples and preparations hereto.

For some of the steps of the here above described process of preparation of the nicotinamide derivatives of formula (I), it can be necessary to protect the potential reactive functions that are not wished to react. In such a case, any compatible protecting radical can be used. In particular methods such as those described by T. W. GREENE (Protective Groups in Organic Synthesis, A. Wiley-lnterscience Publication, 1981) or by McOMIE (Protective Groups in Organic Chemistry, Plenum Press, 1973), can be used.

Also, the nicotinamide derivatives of formula (I) as well as intermediate for the preparation thereof can be purified according to various well-known methods, such as for example crystallization or chromatography.

Thus according to a further embodiment the present invention provides a process for the preparation of a nicotinamide derivative of the formula (I) as described in claim 1 comprising:
(i) reaction of amine (VI) with a suitable compound of the formula R³SO₂LG, where LG is a suitable leaving group; or
(ii) substitution of the leaving group, LG, of the compounds of formula (IX) by an optionally substituted, 3-methylsulphanyl-phenol group; or
(iii) de-protected compounds of formula (V) and subsequently reaction with a suitable acid of formula R³COOH
   wherein formulae (VI), (IX) and (V) are as defined hereinbefore.

The present invention additionally provides compounds of the general formulae (VI), (IX) and (V) as defined hereinbefore.

According to a yet further embodiment the present invention provides processes for the preparation of compounds of general formulae (VI), (IX) and (V) wherein said processes are as illustrated by steps (a), (b) and (c) Route A, steps (d), (c) and (a) Route B and steps (b), (e) and (a) Route C herein.

The nicotinamide derivatives of formula (I) may also be optionally transformed in pharmaceutically acceptable salts. In particular, these pharmaceutically acceptable salts of the nicotinamide derivatives of the formula (I) include the acid addition and the base salts (including disalts) thereof.

Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodie, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, 1-hydroxy-2-naphtoate, 3-hydroxy-2-naphthoate and tosylate saltes.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see Stahl and Wermuth, Handbook of Pharmaceutical Salts: Properties, Selection and Use, Wiley-VCH, Weinheim, Germany (2002).

A pharmaceutically acceptable salt of a nicotinamide derivative of the formula (I) may be readily prepared by mixing together solutions of the nicotinamide derivative of formula (I) and the desired acid or base, as appropriate. The salt may precipitate from solution and be collected by filtration or may be recovered by evaporation of the solvent.

Pharmaceutically acceptable solvates in accordance with the invention include hydrates and solvates wherein the solvent of crystallization may be isotopically substituted, e.g. D2O, d₆-acetone, d₆-DMSO.

Also within the scope of the invention are clathrates, drug-host inclusion complexes wherein, in contrast to the aforementioned solvates, the drug and host are are present in non-stoichiometric amounts. For a review of such complexes, see J Pharm Sci, 64 (8), 1269-1288 by Haleblian (August 1975).

Hereinafter all references to nicotinamide derivatives of formula (I) include references to salts thereof and to solvates and clathrates of compounds of formula (I) and salts thereof.

The invention includes all polymorphs of the nicotinamide derivatives of formula (I).

Also within the scope of the invention are so-called “prodrugs” of the nicotinamide derivatives of formula (I). Thus certain derivatives of nicotinamide derivatives of formula (I) which have little or no pharmacological activity themselves can, when metabolised upon administration into or onto the body, give rise to nicotinamide derivatives of formula (I) having the desired activity. Such derivatives are referred to as “prodrugs”.

Prodrugs in accordance with the invention can, for example, be produced by replacing appropriate functionalities present in the nicotinamide derivatives of formula (I) with certain moieties known to those skilled in the art as “pro-moieties” as described, for example, in “Design of Prodrugs” by H Bundgaard (Elsevier, 1985).

Finally, certain nicotinamide derivatives of formula (I) may themselves act as prodrugs of other nicotinamide derivatives of formula (I).

Nicotinamide derivatives of formula (I) containing one or more asymmetric carbon atoms can exist as two or more optical isomers. Where a nicotinamide derivative of formula (I) contains an alkenyl or alkenylene group, geometric cis/trans (or Z/E) isomers are possible, and where the nicotinamide derivative contains, for example, a keto or oxime group, tautomeric isomerism (‘tautomerism’) may occur. It follows that a single nicotinamide derivative may exhibit more than one type of isomerism.

Included within the scope of the present invention are all optical isomers, geometric isomers and tautomeric forms of the nicotinamide derivatives of formula (I), including compounds exhibiting more than one type of isomerism, and mixtures of one or more thereof.

Cis/trans isomers may be separated by conventional techniques well known to those skilled in the art, for example, fractional crystallisation and chromatography. Conventional techniques for the preparation/isolation of individual stereoisomers include the conversion of a suitable optically pure precursor, resolution of the racemate (or the racemate of a salt or derivative) using, for example, chiral HPLC, or fractional crystallisation of diastereoisomeric salts formed by reaction of the racemate with a suitable optically active acid or base, for example, tartaric acid.

The present invention also includes all pharmaceutically acceptable isotopic variations of a nicotinamide derivative of formula (I). An isotopic variation is defined as one in which at least one atom is replaced by an atom having the same atomic number, but an atomic mass different from the atomic mass usually found in nature.

Examples of isotopes suitable for inclusion in the nicotinamide derivatives of the invention include isotopes of hydrogen, such as ²H and ³H, carbon, such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the nicotinamide derivative of formula (I) isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the nicotinamide derivatives of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the nicotinamide derivatives of formula (I) can generally be prepared by conventional techniques known to those skilled in the art or by processes analogous to those described in the accompanying Examples and Preparations using appropriate isotopic variations of suitable reagents.

According to a further aspect, the present invention concerns mixtures of nicotinamide derivatives of the formula (I), as well as mixtures with or of their pharmaceutically acceptable salts, solvates, polymorphs, isomeric forms and/or isotope forms.

According to the present invention, all the here above mentioned forms of the nicotinamide derivatives of formula (I) except the pharmaceutically acceptable salts (i.e. said solvates, polymorphs, isomeric forms and isotope forms), are defined as “derived forms” of the nicotinamide derivatives of formula (I) in what follows.

The nicotinamide derivatives of formula (I), their pharmaceutically acceptable salts and/or derived forms, are valuable pharmaceutical active compounds, which are suitable for the therapy and prophylaxis of numerous disorders in which the PDE4 enzymes are involved, in particular the inflammatory disorders, allergic disorders, respiratory diseases and wounds.

The nicotinamide derivatives of formula (I) and their pharmaceutically acceptable salts and derived forms as mentioned above can be administered according to the invention to animals, preferably to mammals, and in particular to humans, as pharmaceuticals for therapy or prophylaxis. They can be administered per se, in mixtures with one another or in combination with other drugs, or in the form of pharmaceutical preparations which permit enteral (gastric) or parenteral (non-gastric) administration and which as active constituent contain an efficacious dose of at least one nicotinamide derivative of the formula (I), its pharmaceutically acceptable salts and/or derived forms, in addition to customary pharmaceutically innocuous excipients and/or additives. The term “excipient” is used herein to describe any ingredient other than the compound of the invention. The choice of excipient will to a large extent depend on the particular mode of administration.

The nicotinamide derivatives of formula (I), their pharmaceutically acceptable salts and/or derived forms may be freeze-dried, spray-dried, or evaporatively dried to provide a solid plug, powder, or film of crystalline or amorphous material. Microwave or radio frequency drying may be used for this purpose.

Oral Administration

The nicotinamide derivatives of formula (I) their pharmaceutically acceptable salts and/or derived forms of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The nicotinamide derivatives of formula (I), their pharmaceutically acceptable salts and/or derived forms of the invention may also be used in fast-dissolving, fast-disintegrating dosage forms such as those described in Expert Opinion in Therapeutic Patents, 11 (6), 981-986 by Liang and Chen (2001).

The composition of a typical tablet in accordance with the invention may comprise:

| Ingredient | % w/w |
|---|---|
| Nicotinamide derivative of formula (1) | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

Parenteral Administration

The nicotinamide derivatives of formula (I), their pharmaceutically acceptable salts and/or derived forms of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of nicotinamide derivatives of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

Topical Administration

The nicotinamide derivatives of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus nicotinamide derivatives of formula (I) may be formulated in a more solid form for administration as an implanted depot providing long-term release of the active compound.

Inhaled/Intransal Administration

The nicotinamide derivatives of formula (I) can also be administered intranasally or by inhalation, typically in the form of a dry powder (either alone, as a mixture, for example, in a dry blend with lactose in anhydrous or monohydrate form, preferably monohydrate, mannitol, dextran, glucose, maltose, sorbitol, xylitol, fructose, sucrose or trehalose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1μg to 20 mg of the nicotinamide derivative of formula (I) per actuation and the actuation volume may vary from 1 μl to 100 μl. A typical formulation may comprise a nicotinamide derivative of formula (I), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the nicotinamide derivative of formula (I), a suitable powder base such as lactose or starch and a performance modifier such as I-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff” containing from 1 μg to 4000 μg of the nicotinamide derivative of formula (I). The overall daily dose will typically be in the range 1 μg to 20 mg which may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release. Sustained or controlled release can be obtained by using for example poly(D,L-lactic-co-glycolic acid).

Flavouring agents, such as methol and levomethol and/or sweeteners such as saccharing or saccharin sodium can be added to the formulation.

Rectal/Intravaginal Administration

The nicotinamide derivatives of formula (I) may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

Ocular/Andial Administration

The nicotinamide derivatives of formula (I) may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

Enabling Technologies

The nicotinamide derivatives of formula (I) may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Dosage

For administration to human patients, the total daily dose of the nicotinamide derivatives of formula (I) is typically in the range 0.001 mg/kg to 100 mg/kg depending, of course, on the mode of administration. The total daily dose may be administered in single or divided doses. The physician will readily be able to determine doses for subjects depending on age, weight, health state and sex or the patient as well as the severity of the disease.

According to another embodiment of the present invention, the nicotinamide derivatives of the formula (I), their pharmaceutically acceptable salts and/or their derived forms, can also be used as a combination with one or more additional therapeutic agents to be co-administered to a patient to obtain some particularly desired therapeutic end result. The second and more additional therapeutic agents may also be a nicotinamide derivatives of the formula (I), their pharmaceutically acceptable salts and/or their derived forms, or one or more PDE4 inhibitors known in the art. More typically, the second and more therapeutic agents will be selected from a different class of therapeutic agents.

As used herein, the terms “co-administration”, “co-administered” and “in combination with”, referring to the nicotinamide derivatives of formula (I) and one or more other therapeutic agents, is intended to mean, and does refer to and include the following:
simultaneous administration of such combination of nicotinamide derivative(s) and therapeutic agent(s) to a patient in need of treatment, when such components are formulated together into a single dosage form which releases said components at substantially the same time to said patient,
substantially simultaneous administration of such combination of nicotinamide derivative(s) and therapeutic agent(s) to a patient in need of treatment, when such components are formulated apart from each other into separate dosage forms which are taken at substantially the same time by said patient, whereupon said components are released at substantially the same time to said patient,
sequential administration of such combination of nicotinamide derivative(s) and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of nicotinamide derivative(s) and therapeutic agent(s) to a patient in need of treatment, when such components are formulated together into a single dosage form which releases said components in a controlled manner whereupon they are concurrently, consecutively, and/or overlappingly administered at the same and/or different times by said patient.

Suitable examples of other therapeutic agents which may be used in combination with the nicotinamide derivatives of the formula (I), their pharmaceutically acceptable salts and/or their derived forms include, but are by no mean limited to:
(a) 5-Lipoxygenase (5-LO) inhibitors or 5-lipoxygenase activating protein (FLAP) antagonists,
(b) Leukotriene antagonists (LTRAs) including antagonists of LTB4, LTC4, LTD4, and LTE4,
(c) Histaminic receptor antagonists including H1, H3 and H4 antagonists,
(d) α1- and α2-adrenoceptor agonist vasoconstrictor sympathomimetic agents for decongestant use,
(e) Muscarinic M3 receptor antagonists or anticholinergic agents,
(f) β2-adrenoceptor agonists,
(g) Theophylline,
(h) Sodium cromoglycate,
(i) COX-1 inhibitors (NSAIDs) and COX-2 selective inhibitors,
(j) Oral or inhaled Glucocorticosteroids,
(k) Monoclonal antibodies active against endogenous inflammatory entities,
(l) Anti-tumor necrosis factor (anti-TNF-a) agents,
(m) Adhesion molecule inhibitors including VLA4 antagonists,
(n) Kinin-B1- and B2-receptor antagonists,
(o) Immunosuppressive agents,
(p) Inhibitors of matrix metalloproteases (MMPs),
(q) Tachykinin NK1, NK2 and NK3 receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFkb pathway, e.g. IKK inhibitors,
(w) Agents that can be classed as mucolytics or anti-tussive,
(x) antibiotics, and
(y) p38 MAP kinase inhibitors

According to the present invention, combination of the nicotinamide derivatives of formula (I) with:
muscarinic M3 receptor agonists or anticholinergic agents including in particular ipratropium salts, namely bromide, tiotropium salts, namely bromide, oxitropium salts, namely bromide, perenzepine, and telenzepine,
β2-adrenoceptor agonists including albutarol, salbutamol, formoterol and salmeterol,
p38 MAP kinase inhibitors,
H3 antagonists,
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, and mometasone furoate,
or adenosine A2a receptor agonists, are preferred.

It is to be appreciated that all references herein to treatment include curative, palliative and prophylactic treatment. The description which follows concerns the therapeutic applications to which the nicotinamide derivatives of formula (I) may be put.

The nicotinamide derivatives of formula (I) inhibit the PDE4 isozyme and thereby have a wide range of therapeutic applications, as described further below, because of the essential role, which the PDE4 family of isozymes plays in the physiology of all mammals. The enzymatic role performed by the PDE4 isozymes is the intracellular hydrolysis of adenosine 3′,5′-monophosphate (cAMP) within pro-inflammatory leukocytes. cAMP, in turn, is responsible for mediating the effects of numerous hormones in the body, and as a consequence, PDE4 inhibition plays a significant role in a variety of physiological processes. There is extensive literature in the art describing the effects of PDE inhibitors on various inflammatory cell responses, which in addition to cAMP increase, include inhibition of superoxide production, degranulation, chemotaxis and tumor necrosis factor (TNF) release in eosinophils, neutrophils and monocytes.

Therefore, a further aspect of the present invention relates to the use of the nicotinamide derivatives of formula (I), their pharmaceutically acceptable salts and/or derived forms, in the treatment of diseases, disorders, and conditions in which the PDE4 isozymes are involved. More specifically, the present invention also concerns the use of the nicotinamide derivatives of formula (I), their pharmaceutically acceptable salts and/or derived forms, in the treatment of diseases, disorders, and conditions selected from the group consisting of:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy
pneumoconiosis of whatever type, etiology, or pathogenesis, in particular pneumoconiosis that is a member selected from the group consisting of aluminosis or bauxite workers'disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,
seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,
rheumatoid arthritis of whatever type, etiology, or pathogenesis, in particular rheumatoid arthritis that is a member selected from the group consisting of acute arthritis, acute gouty arthritis, chronic inflammatory arthritis, degenerative arthritis, infectious arthritis, Lyme arthritis, proliferative arthritis, psoriatic arthritis and vertebral arthritis,
gout, and fever and pain associated with inflammation,
an eosinophil-related disorder of whatever type, etiology, or pathogenesis, in particular an eosinophil-related disorder that is a member selected from the group consisting of eosinophilia, pulmonary infiltration eosinophilia, Loffler's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrotizing vasculitis,
atopic dermatitis, allergic dermatitis, contact dermatitis, or allergic or atopic eczema,
urticaria of whatever type, etiology, or pathogenesis, in particular urticaria that is a member selected from the group consisting of immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria and papular urticaria,
conjunctivitis of whatever type, etiology, or pathogenesis, in particular conjunctivitis that is a member selected from the group consisting of actinic conjunctivitis, acute catarrhal conjunctivitis, acute contagious conjunctivitis, allergic conjunctivitis, atopic conjunctivitis, chronic catarrhal conjunctivitis, purulent conjunctivitis and vernal conjunctivitis,
uveitis of whatever type, etiology, or pathogenesis, in particular uveitis that is a member selected from the group consisting of inflammation of all or part of the uvea, anterior uveitis, iritis, cyclitis, iridocyclitis, granulomatous uveitis, nongranulomatous uveitis, phacoantigenic uveitis, posterior uveitis, choroiditis; and chorioretinitis,
psoriasis;
multiple sclerosis of whatever type, etiology, or pathogenesis, in particular multiple sclerosis that is a member selected from the group consisting of primary progressive multiple sclerosis and relapsing remitting multiple sclerosis,
autoimmune/inflammatory diseases of whatever type, etiology, or pathogenesis, in particular an autoimmune/inflammatory disease that is a member selected from the group consisting of autoimmune hematological disorders, hemolytic anemia, aplastic anemia, pure red cell anemia, idiopathic thrombocytopenic purpura, systemic lupus erythematosus, polychondritis, scleroderma, Wegner's granulomatosis, dermatomyositis, chronic active hepatitis, myasthenia gravis, Stevens-Johnson syndrome, idiopathic sprue, autoimmune inflammatory bowel diseases, ulcerative colitis, endocrin opthamopathy, Grave's disease, sarcoidosis, alveolitis, chronic hypersensitivity pneumonitis, primary biliary cirrhosis, juvenile diabetes or diabetes mellitus type I, keratoconjunctivitis sicca, epidemic keratoconjunctivitis, diffuse interstitial pulmonary fibrosis or interstitial lung fibrosis, idiopathic pulmonary fibrosis, cystic fibrosis, glomerulonephritis with and without nephrotic syndrome, acute glomerulonephritis, idiopathic nephrotic syndrome, minimal change nephropathy, inflammatory/hyperproliferative skin diseases, benign familial pemphigus, pemphigus erythematosus, pemphigus foliaceus, and pemphigus vulgaris,
prevention of allogeneic graft rejection following organ transplantation,
inflammatory bowel disease (IBD) of whatever type, etiology, or pathogenesis, in particular inflammatory bowel disease that is a member selected from the group consisting of collagenous colitis, colitis polyposa, transmural colitis, ulcerative colitis and Crohn's disease (CD),
septic shock of whatever type, etiology, or pathogenesis, in particular septic shock that is a member selected from the group consisting of renal failure, acute renal failure, cachexia, malarial cachexia, hypophysial cachexia, uremic cachexia, cardiac cachexia, cachexia suprarenalis or Addison's disease, cancerous cachexia and cachexia as a consequence of infection by the human immunodeficiency virus (HIV),
liver injury,
pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension,
bone loss diseases, primary osteoporosis and secondary osteoporosis,
central nervous system disorders of whatever type, etiology, or pathogenesis, in particular a central nervous system disorder that is a member selected from the group consisting of depression, Alzheimers disease, Parkinson's disease, learning and memory impairment, tardive dyskinesia, drug dependence, arteriosclerotic dementia and dementias that accompany Huntington's chorea, Wilson's disease, paralysis agitans, and thalamic atrophies,
infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex,
yeast and fungus infections wherein said yeast and fungi are sensitive to upregulation by TNF-α or elicit TNF-α production in their host, e.g., fungal meningitis, particularly when administered in conjunction with other drugs of choice for the treatment of systemic yeast and fungus infections, including but are not limited to, polymixins, e.g. Polymycin B, imidazoles, e.g. clotrimazole, econazole, miconazole, and ketoconazole, triazoles, e.g. fluconazole and itranazole as well as amphotericins, e.g. Amphotericin B and liposomal Amphotericin B,
ischemia-reperfusion injury, ischemic heart disease, autoimmune diabetes, retinal autoimmunity, chronic lymphocytic leukemia, HIV infections, lupus erythematosus, kidney and ureter disease, urogenital and gastrointestinal disorders and prostate diseases,
reduction of scar formation in the human or animal body, such as scar formation in the healing of acute wounds, and
psoriasis, other dermatological and cosmetic uses, including antiphlogistic, skin-softening, skin elasticity and moisture-increasing activities.

According to one aspect the present invention relates in particular to the treatment of a respiratory disease, such as adult respiratory distress syndrome (ARDS), bronchitis, chronic obstructive pulmonary disease (COPD), cystic fibrosis, asthma, emphysema, bronchiectasis, sinusitis and rhinitis.

According to another aspect the present invention relates in particular to the treatment of gastrointestinal (GI) disorders, in particular inflammatory bowel diseases (IBD) such as Crohn's disease, ileitis, collagenous colitis, colitis polyposa, transmural colitis and ulcerative colitis.

According to a further aspect the present invention relates in particular to the reduction of scars formation.

A still further aspect of the present invention also relates to the use of the nicotinamide derivatives of formula (I), their pharmaceutically acceptable salts and/or derived forms, for the manufacture of a drug having a PDE4 inhibitory activity. In particular, the present inventions concerns the use of the nicotinamide derivatives of formula (I), their pharmaceutically acceptable salts and/or derived forms, for the manufacture of a drug for the treatment of inflammatory, respiratory, allergic and scar-forming diseases, disorders, and conditions, and more precisely for the treatment of diseases, disorders, and conditions that are listed above.

As a consequence, the present invention provides a particularly interesting method of treatment of a mammal, including a human being, with a PDE4 inhibitor including treating said mammal with an effective amount of a nicotinamide derivative of formula (I), its pharmaceutically acceptable salts and/or derived forms. More precisely, the present invention provides a particularly interesting method of treatment of a mammal, including a human being, to treat an inflammatory, respiratory, allergic and scar-forming disease, disorder or condition, including treating said mammal with an effective amount of a nicotinamide derivative of formula (I), its pharmaceutically acceptable salts and/or derived forms.

The following examples illustrate the preparation of the nicotinamide derivatives of the formula (I):

Where Preparations or Examples are described as being effected by a method “similar to” another method this means that minor differences in the practical method may exist, such as for example use of recrystallisation rather than column chromatography in the purification stage or use of alternative solvents in separation phase. However such minor differences are considered to be within the common general knowledge and experimental experience of the skilled chemist when approaching such reactions.

### PREPARATION 1

### 2-Chloro-5-fluoro Nicotinic Acid

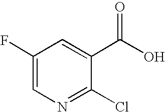

Ethyl-2-chloro-5-fluoro-nicotinoate (50.4 g, 0.247 mol) (can be prepared according to the method of J. Med. Chem., 1993, 36(18), 2676-88, page 2684, column 2, 3^{rd} example, ethyl-2-chloro-5-fluoropyridine-3-carboxylate) was dissolved in tetrahydrofuran (350 mL) and a 2M aqueous solution of lithium hydroxide (247 mL, 0.495 mol) added. The reaction mixture was stirred at room temperature for 3 days. The pH of the solution was reduced to pH1 by addition of 6M hydrochloric acid and then extracted with dichloromethane (3×). The combined extracts were dried (MgSO₄) and the solvent evaporated under reduced pressure to give a solid which was triturated with diethyl ether and then dried to give the title compound as a white solid, 40.56 g.

¹H NMR (DMSO-d₆, 400 MHz) δ: 8.20 (s, 1H), 8.62 (s, 1H).

LRMS: m/z ES⁺ 174 [MH]⁺

### PREPARATION 2

### Trans-N-tert-butyl (4-hydroxy-cyclohexyl)-carbamate

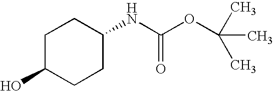

Trans-4-aminocyclohexanol (100 g, 0.87 mol) was added to acetonitrile (1 L) with stirring followed by di-tert-butyl dicarbonate (208 g, 0.96 mol) in portions over 1 hour. The reaction was stirred at room temperature for 18 hours, the resulting precipitate filtered off, and washed with ethyl acetate:hexane (1:3, 250 mL), then hexane (250 mL) and dried to afford the title compound as a white solid, 166.9 g.

m.p.-167-170° C.

### PREPATRATION 3

### Trans-Methanesulphonic Acid 4-tert-butoxycarbonylamino-cyclohexyl Ester

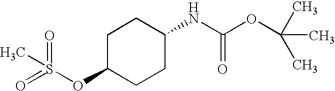

A solution of mesyl chloride (122.4 g, 1.07 mol) in dichloromethane (400 mL) was added dropwise over 45 minutes to an ice-cooled solution of the alcohol from preparation 2 (200 g, 0.93 mol) and triethylamine (112.8 g, 1.115 mol) in dichloromethane (1 L). The reaction was stirred for 15 minutes, then allowed to warm to room temperature over 1 hour. The mixture was washed with water (3×1.5 L), and then stirred with silica (100 mL, Merck 60H). This mixture was filtered and the filtrate concentrated under reduced pressure to approx quarter volume. Hexane (500 mL) was added, the mixture was cooled to 0° C., and the resulting solid filtered off, dried and recrystallised from ethyl acetate to give the title compound, 221.1 g.

m.pt.-146-148° C.

### PREPARATION 4

### Syn-(4-Azido-cyclohexyl)-carbamic Acid Tert-butyl Ester

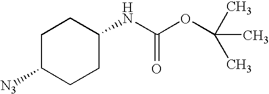

Sodium azide (25.5 g, 0.39 mol) was added to a solution of the mesylate from preparation 3 (100 g, 0.34 mol) in N,N-dimethylformamide (500 mL) and the reaction slowly warmed to 80° C., and stirred for a further 24 hours at this temperature. Ice/water (1 L) was added slowly to the cooled reaction, and the resulting precipitate was filtered off, washed with water and dried. The solid was dissolved in ethyl acetate (200 mL), the solution washed with water, dried (MgSO₄) and evaporated under reduced pressure. The residual solid was recrystallised from hexane to afford the title compound as a white solid, 50.8 g.

m.pt. 79-81° C.

### PREPARATION 5

### Syn-tert-Butyl 4-aminocyclohexvlcarbamate

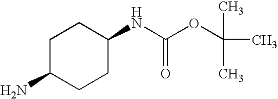

5% Palladium on charcoal (5 g) was mixed with toluene (10 mL) and was added to the product from preparation 4 (170 g, 0.71 mol) in methanol (400 mL). The mixture was hydrogenated (80 atmospheres) at room temperature for 18 hours and then filtered. The solvent was evaporated under reduced pressure and the residue was triturated with ethyl acetate (50 mL) and then with hexane (200 mL). The solid obtained was isolated by filtration, dissolved in ethyl acetate (600 mL) and filtered through Celite®. The filtrate was concentrated under reduced pressure to give a slush that was diluted with hexane (300 mL). The solid obtained was isolated by filtration and was washed with ethyl acetate in hexane (20:80). The mother liquors were combined and evaporated under reduced pressure, and the residue was purified by chromatography on silica gel using ethyl acetate and then methanol as eluant. The material obtained was crystallised from ethyl acetate and hexane and combined with the first crop to give the title compound as a white solid, 76.0 g.

M.pt. 88-90° C.

¹H NMR (CDCl₃, 400 MHz) δ: 1.41 (s, 9H), 1.52-1.77 (m, 8H), 1.82 (m, 1H), 1.97 (m, 1H), 2.61 (m, 1H), 3.62 (m, 1H), 4.59 (m, 1H).

LRMS: m/z ES⁺ 215 [MH]⁺

### PREPARATION 6

### Syn-{4-[(2-Chloro-5-fluoropyridine-3-carbonyl)amino]-cyclohexyl}-carbamic Acid tert-butyl Ester

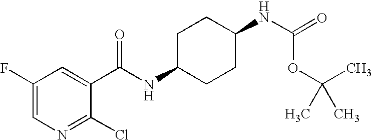

Oxalyl chloride (8 mL, 90 mmol) was added over 10 minutes to an ice-cooled suspension of the acid from preparation 1 (10 g, 57 mmol) and N,N-dimethylformamide (5 drops) in dichloromethane (200 mL). The suspension was then stirred at room temperature for 3 hours, and concentrated under reduced pressure. The residue was azeotroped with dichloromethane to give the intermediate acid chloride as a white solid. This white solid was dissolved in dichloromethane (200 mL), the solution cooled in a water bath, then N-diisopropylethylamine (20 mL, 115 mmol) and the amine from preparation 5 (13.4 g, 62 mmol) were added. The reaction mixture was stirred for 18 hours, diluted with dichloromethane (100 mL) and washed sequentially with 10% citric acid solution, saturated sodium bicarbonate solution (×2), water and then brine. The organic solution was dried (MgSO₄) and evaporated under reduced pressure to afford the title compound as yellow foam, 20.2 g.

¹H NMR (400 MHz, CDCl₃) δ:1.27 (s, 9H), 1.76 (m, 2H), 1.86 (m, 6H), 3.64 (m, 1H), 4.16 (m, 1H), 4.54 (m, 1H), 6.67 (s, 1H), 7.80 (m, 1H), 8.33 (d, 1H).

LRMS: m/z ES⁺ 394 [MNa]⁺

### PREPATRATION 7

### 1-Fluoro-4-methoxy-2-methylsulphanyl-benzene

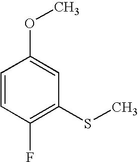

1,2-Difluoro-4-methoxy-benzene (100 mg, 0.69 mmol) and sodium methan-ethiolate (148 mg, 2.08 mmol) were dissolved in N,N-dimethylformamide (2 mL) and the reaction mixture stirred at 60° C. for 18 hours. Additional sodium methanethiolate (99 mg, 139 mmol) was added and the reaction mixture was heated to 100° C. for 18 hours. The reaction mixture was diluted with water and extracted with ether (×2). The ether extracts were washed with water (×2), dried (MgSO₄) and then concentrated under reduced pressure. The residue was taken up in a pentane:ether 1:1 solution (2 mL) and filtered through a plug of silica in a pipette, and then washed through with a pentane:ether 1:1 solution (5 mL). The solution was concentrated under reduced pressure to yield the title product as a colourless oil, 135 mg.

¹H NMR (CDCl₃, 300 MHz) δ: 2.45 (s, 3H), 3.80 (s, 3H), 6.65 (dd, 1H), 6.80 (dd, 1H), 6.95 (dd, 1H).

### PREPARATION 8

### 4-Fluoro-3-methylsulphanyl-phenol

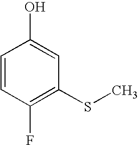

Boron tribromide (2.5 mL, 2.5 mmol) was added to a solution of the ether of preparation 7 (118.3 mg, 0.69 mmol) in dichloromethane (10 mL) at 0° C. under nitrogen and the reaction mixture was stirred at 0° C. for 4 hours. The reaction was then stirred for a further 18 hours at room temperature. The reaction mixture was quenched with sodium carbonate solution (10 mL) and stirred for 1 hour. The mixture was acidified with 2M hydrochloric acid the layers were then separated and the organic phase was concentrated under reduced pressure. The crude product was taken up in a 1:1 solution of ether:pentane solution (2 mL) and the solution was then filtered through a plug of silica in a pipette. The residue was washed with additional ether:pentane solution 1:1 (5 mL) and concentrated under reduced pressure to yield the title product as a yellow oil, 110 mg.

¹H NMR (CDCl₃, 400 MHz) δ: 2.45 (s, 3H), 6.57 (m, 1H), 6.72 (m, 1H), 6.90 (t, 1H).

LRMS: m/z ES⁻ 157 [M-H]³¹

### PREPARATION 9

### 5-Fluoro-2-(3-methylsulphanyl-phenoxy)-nicotinic Acid Ethyl Ester

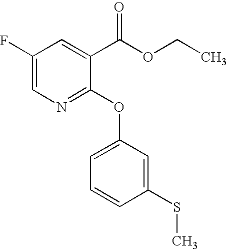

A solution of ethyl-2-chloro-5-fluoro-nicotinate (as prepared according to the method described in J. Med. Chem., 1993, 36(18), 2676-88, page 2684, column 2, 3^{rd} compound, Ethyl 2-chloro-5-fluoropyridine-3-carboxylic acid) and 3-methylsulphanyl-phenol (20 g, 0.143 mol) (prepared according to the method described in WO 98/45268, page 68, preparation 61) in dioxane (300 mL) was treated with caesium carbonate (46.5 g, 0.143 mol) at room temperature. The reaction mixture was heated to 100° C. and stirred for 48 hours. The reaction mixture was concentrated under reduced pressure and the residue taken up in water (600 mL) and extracted with ethyl acetate (3×250 mL). The organic layers were combined, washed with brine (200 mL), dried (MgSO₄) and concentrated under reduced pressure. The residue was purified by column chromatography on silica gel eluting with dichloromethane:toluene (99.75:0.25 to 99.5:0.5) to yield the title product as a yellow oil, 27.1 g.

¹H NMR (CDCl₃, 400 MHz) δ: 1.37 (t, 3H), 2.23 (s, 3H), 4.40 (q, 2H), 6.84 (m, 1H) 7.01 (m, 1H) 7.08 (m, 1H), 7.26 (m, 1H), 7.98 (m, 1H), 8.13 (m, 1H).

LRMS: m/z APCl⁺ 308 [MH]⁺

### PREPARATION 10

### 5-Fluoro-2-(3-methylsulphanyl-phenoxy)-nicotinic Acid

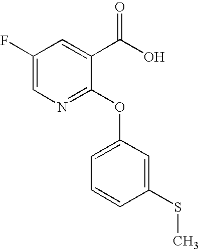

The ester of preparation 9 (27.1 g, 88.2 mmol) was dissolved in tetrahydrofuran (300 mL) and the solution treated with a 1M aqueous solution of lithium hydroxide (220 mL, 220 mmol). The reaction mixture was stirred at room temperature for 2 hours. The reaction was concentrated under reduced pressure to remove the tetrahydrofuran and the aqueous was cooled to 0° C. before being acidified to pH 1 with hydrochloric acid. The resulting pink precipitate was removed by filtration and washed with iced water. The solid was dissolved in dichloromethane (800 mL) and washed with acidified brine solution (200 mL). The organic layer was separated, dried (MgSO₄) and concentrated under reduced pressure. The residue was triturated with toluene to yield the title product as a white solid, 22.13 g (90%).

¹H NMR (CD₃OD, 400 MHz) δ: 2.43 (s, 3H), 6.83 (m, 1H), 7.01 (m, 1H), 7.06 (m, 1H), 7.25 (m,1H), 8.03 (m, 2H).

LRMS: m/z APCl⁺ 280 [MH]⁺

### PREPARATION 11

### Syn-(4-{[5-Fluoro-2-(3-methylsulphanyl-phenoxy)-pyridine-3-carbonyl]-amino}-cyclohexyl)-carbamic Acid Tert-butyl Ester

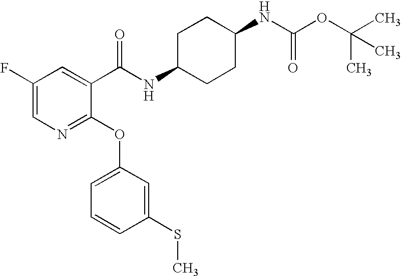

The acid of preparation 10 (5 g, 17.9 mmol) and N,N-dimethylformamide (5 drops) were dissolved in dichloromethane (100 mL) and the reaction mixture cooled to 0° C. The mixture was treated dropwise with oxalyl chloride (3.1 mL, 35.8 mmol) over 15 minutes and then stirred at room temperature for 2 hours. The reaction mixture was concentrated under reduced pressure and the residue taken up in dichloromethane (100 mL). The solution was cooled to 0° C. and treated with triethylamine (7.5 mL, 54 mmol) and the amine of preparation 5 (4.2 g, 19.6 mmol). The reaction was allowed to warm to room temperature and was stirred at room temperature for 48 hours. The reaction mixture was diluted with dichloromethane (100 mL) and washed with water (70 mL), 10% citric acid solution (2×70 mL), saturated sodium hydrogencarbonate solution (2×70 mL) and water (70 mL). The organic layer was dried (MgSO₄) and concentrated under reduced pressure to yield the title product, 8.0 g. ¹H NMR (CDCl₃, 400 MHz) δ: 1.40 (s, 9H), 1.53 (m, 2H), 1.68 (m, 2H), 1.77 (m, 4H), 2.46(s, 3H), 3.60 (m, 1H), 4.18(m, 1H), 4.37 (m, 1H), 6.88 (m, 1H), 7.02 (m, 1H), 7.17 (m, 1H), 7.37 (m, 1H), 7.93 (m, 1H), 8.06 (m, 1H), 8.36 (m, 1H).

LRMS: m/z ES⁺ 476 [MH]⁺

### PREPARATION 12

### Syn-(4-{[5-Fluoro-2-(3-methylsulphanyl-phenoxy)-pyridine-3-carbonyl]-amino}-cyclohexyl)-carbamic Acid Tert-butyl Ester

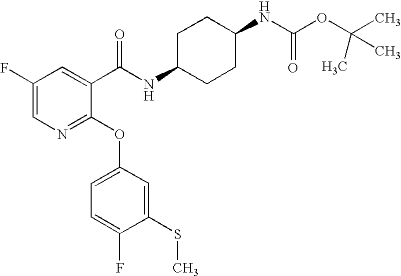

The chloro compound of preparation 6 (6.4 g, 17.2 mmol), the phenol of preparation 8 (3.0 g, 19.0 mmol) and caesium carbonate (11.2 g, 34.4 mmol) were dissolved in dioxane (200 mL) and the reaction mixture refluxed for 72 hours. The reaction mixture was cooled to room temperature and concentrated under reduced pressure. The residue was taken up in ethyl acetate and water and the layers separated. The aqueous was extracted with ethyl acetate, the organics were combined, dried (MgSO₄) and concentrated under reduced pressure. The residue was purified by column chromatography on silica gel eluting with dichloromethane:methanol (99.5:0.5 to 98:2) to yield the title product as a pale yellow foam, 6.31 g.

¹H NMR (CDCl₃, 400 MHz) δ: 1.42-1.84 (m, 17H), 2.48 (s, 3H), 3.62 (m, 1H), 4.18(m, 1H), 4.21 (m, 1H), 6.82 (m, 1H), 7.01 (m, 1H), 7.22 (t, 1H), 7.88 (m, 8.04 (m,1H), 8.36 (m,1H).

LRMS: m/z ES⁺ 516 [MNa]⁺

### PREPARATION 13

### Syn-N-(4-Amino-cyclohexyl)-5-fluoro-2-(3-methylsulphanyl-phenoxy)-nicotinamide Hydrochloride

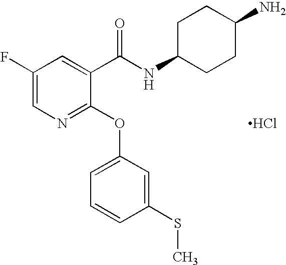

The protected amine of preparation 11 (8.0 g, 16.8 mmol) was dissolved in dioxane (50 mL) and the solution treated with a 4M solution of hydrochloric acid in dioxane (25 mL). The reaction mixture was stirred at room temperature for 5 hours before being concentrated under reduced pressure and azeotroped with ethyl acetate and dichloromethane to yield the title product, 5.0 g.

¹H NMR (CD₃OD, 400 MHz) δ: 1.67 (m, 2H), 1.80-2.01 (m, 6H), 2.45 (s, 3H), 3.24 (m, 1H), 4.14 (m, 1H), 6.92 (m, 1H), 7.09 (m, 1H), 7.17 (m, 1H), 7.35 (t, 1H) (m, 2H).

LRMS: m/z ES⁺ 376 [MH]⁺

### PREPARATION 14

### Syn-N-(4-Amino-cyclohexyl)-5-fluoro-2-(3-methylsulphanyl-phenoxy)-nicotinamide Hydrochloride

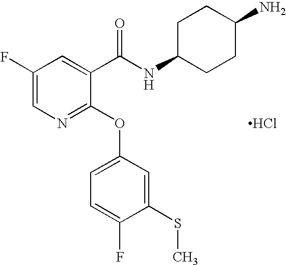

The protected amine of preparation 12 (6.31 g, 12.8 mmol) was dissolved in dichloromethane (100 mL) and the solution cooled to 0° C. Hydrogen chloride gas was bubbled through the solution for 1 hour and the mixture was then allowed to stir for a further hour. Hydrogen chloride gas was again bubbled through the reaction for 1 hour and the reaction mixture allowed to stir at room temperature for 48 hours. The reaction was concentrated under reduced pressure and the residue was triturated with ethyl acetate to yield the title product as a pale pink solid, 4.70 g.

¹H NMR (DMSO-D₆, 400 MHz) δ: 1.60-1.84 (m, 8H), 2.45 (s, 3H), 3.11 (m, 1H), 3.92 (m, 1H), 7.03 (m, 1H), 7.22 (m, 2H), 7.92-8.04 (m, 4H), 8.21 (m, 1H), 8.31 (m, 1H).

LRMS: m/z APCl⁺ 394 [MH]⁺

### PREPARATION 15

5-Methoxy-benzo[1,2,5]thiadiazole-4-sulphonyl Chloride

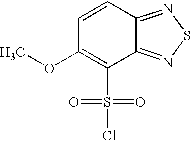

5-Methoxy-benzo[1,2,5]thiadiazole (500 mg, 3 mmol) was added portion-wise over 10 minutes to ice-cold chlorosulphonic acid (1.0 mL), and once addition was complete, the reaction was heated to 100° C. for 1 hour. The cooled solution was poured into ice-water (15 mL), and the resulting precipitate was filtered off and dried to give the title compound as a beige solid, 535 mg.

¹H NMR (CDCl₃, 400 MHz) 67 : 4.28 (s, 3H), 7.64 (d, 1H), 8.36 (d, 1H).

### PREPARATION 16

### Syn-5-Fluoro-N-[4-(5-methoxy-benzo[1,2,5]thiadiazole-4-sulphonylamino)-cyclohexyl]-2-(3-methylsulphanyl-phenoxy)-nicotinamide

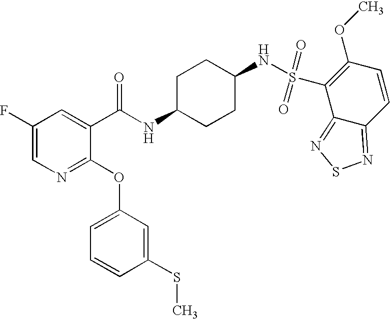

A mixture of the amine from preparation 13 (200 mg, 0.49 mmol), the sulphonyl chloride from preparation 15 (193 mg, 0.73 mmol) and triethylamine (0.2 mL, 1.4 mmol) in dichloromethane (10 mL) was stirred at room temperature for 24 hours. The reaction was diluted with 10% citric acid solution, the layers separated, and the organic phase concentrated under reduced pressure. The residual oil was crystallised from isopropyl acetate/dichloromethane and dried to give the title compound as a beige solid, 254 mg.

¹H NMR (DMSO-d₆, 400 MHz) δ: 1.39-1.68 (m, 8H), 2.51 (s, 3H), 3.18 (m, 1H), 3.75 (m, 1H), 4.04 (s, 3H), 6.92 (m, 1H), 7.09 (m, 2H), 7.35 (m, 2H), 7.83 (d, 1H), 7.97 (m, 1H), 8.21 (m, 2H), 8.34 (d, 1H). LRMS: m/z ES⁺ 626 [MNa]⁺

Microanalysis found; C, 51.27; H, 4.52; N, 11.27. C₂₆H₂₆FN₅O₅S₃ requires C, 51.73; H, 4.34; N, 11.60%.

### PREPARATION 17

### Syn-5-Fluoro-2-(4-fluoro-3-methylsulphanyl-phenoxy)-N-[4-(2-methoxy-5-methyl-benzenesulphonylamino)-cyclohexyl]-nicotinamide

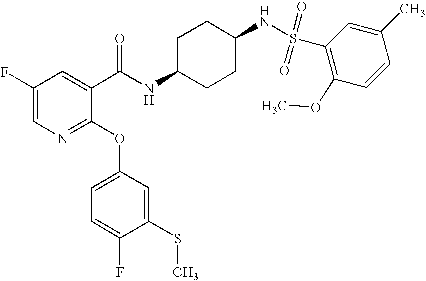

The title compound was obtained as a white solid in 74% yield, from the amine from preparation 14 and 2-methoxy-5-methylbenzene sulphonyl chloride, following the procedure described in preparation 16, except, the product was crystallised from methanol.

¹H NMR (DMSO-d₆, 400 MHz) δ: 1.39-1.58 (m, 6H), 1.60-1.72 (m, 2H), 2.25 (s, 3H), 2.45 (s, 3H), 3.07 (m, 1H), 3.79 (m, 4H), 7.01 (m, 3H), 7.19 (m, 1H), 7.22 (t, 1H), 7.36 (d, 1H), 7.51 (s, 1H), 7.98 (m, 1H), 8.21 (m, 2H).

LRMS: m/z ES⁺ 600 [MNa]⁺

Microanalysis found; C, 55.98; H, 5.04; N, 7.32. C₂₇H₂₉F₂N₃O₅S₂ requires C, 56.14; H, 5.06; N, 7.27%.

### PREPARATION 18

### Syn-5-Fluoro-2-(3-methylsulphanyl-phenoxy)-N-[4-(2-methoxy4-methyl-benzenesulphonylamino)-cyclohexyl]-nicotinamide

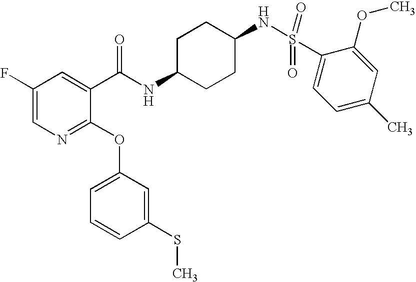

A solution of the amine from preparation 13 (200 mg, 0.49 mmol), triethylamine (0.34 mL, 2.4 mmol) and 6-methoxy-m-toluenesulphonyl chloride (139 mg, 0.63 mmol) in dichloromethane (10 mL) was stirred at reflux for 3 hours, and a further 72 hours at room temperature. The reaction was washed with water, and the organic solution evaporated under reduced pressure. The residue was crystallised from methanol to give the title compound as a white solid, 222 mg.

¹H NMR (DMSO-d₆, 400 MHz) δ: 1.37-1.52 (m, 6H), 1.65 (m, 2H), 2.34 (s, 3H), 2.45 (s, 3H), 3.02 (m, 1H), 3.78 (m, 1H), 3.81 (s, 3H), 6.83 (m, 1H), 6.94 (m, 3H), 7.11 (m, 2H), 7.35 (t, 1H), 7.58 (d, 1H), 7.99 (m, 1H), 8.20 (m, 2H).

LRMS: m/z ES⁺ 582 [MNa]⁺

Microanalysis found; C, 57.49; H, 5.49; N, 7.32. C₂₇H₃₀FN₃O₅S₂; 0.1 H₂O requires C, 57.76; H, 5.42; N, 7.48%.

### EXAMPLES 1 to 9

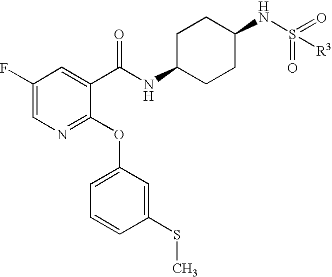

A solution of the amine from preparation 13 (691 mg, 1.68 mmol) and triethylamine (339 mg, 3.36 mmol) in dichloromethane (14 mL) was prepared. 250 μL of this solution was added to the appropriate sulphonyl chloride (30 μmol) and the reactions shaken at room temperature for 48 hours. The reactions were washed with water (250 μL) and the mixtures filtered through a hydrophobic filter, washing through with additional dichloromethane (250 μL). The organic solutions were evaporated under reduced pressure and the residue purified by reverse phase HPLC using a Phenomenex Luna C18, (150*10 mm, 10 μm) column, a flow rate of 8 mLmin⁻¹, detection at 225 nm, and a gradient elution as shown below:

| Time(min) | % acetonitrile | % Diethylamine in water(0.1% v/v) |
|---|---|---|
| 0.0-0.5 | 5 | 95 |
| 0.5-0.6 | 5-10 | 95-90 |
| 0.6-7.0 | 10-95 | 90-5 |
| 7.0-8.5 | 95 | 5 |

| | | | Retention |
|---|---|---|---|
| Example | R³ | LRMS: m/z ES⁻ | Time (min) |
| 1 | 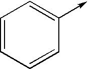 | 513.68 [M − H]⁻ | 7.034 (6.89-7.178) |
| 2 | 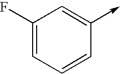 | 531.58 [M − H]⁻ | 7.22 (7.099-7.341) |
| 3 | 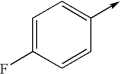 | 532.32 [M − H]⁻ | 7.198 (7.055-7.341) |
| 4 | 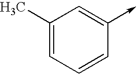 | 527.77 [M − H]⁻ | 7.375 (7.253-7.496) |
| 5 | 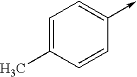 | 527.59 [M − H]⁻ | 7.421 (7.288-7.553) |
| 6 | 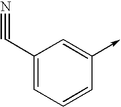 | 539.26 [M − H]⁻ | 6.861 (6.739-6.983) |
| 7 | 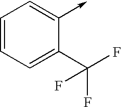 | 582.22 [M − H]⁻ | 7.507 (7.385-7.496) |
| 8 | 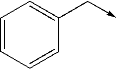 | 527.59 [M − H]⁻ | 7.068 (6.935-7.2) |
| 9 | 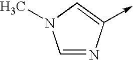 | 517.96 [M − H]⁻ | 5.773 (5.66-5.886) |

### EXAMPLE 10

### Syn-N-[4-(3,5-Dimethyl-1H-pyrazole-4-sulphonylamino)-cyclohexyl]-5-fluoro-2 -(3-methylsulphanyl-phenoxy)-nicotinamide

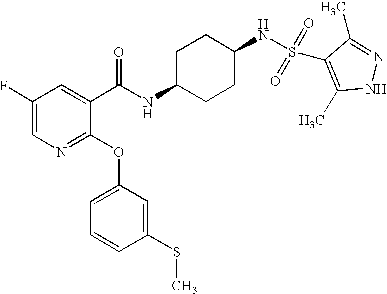

A mixture of the amine from preparation 13 (150 mg, 0.36 mmol), 3,5-dimethyl-pyrazole-4-sulphonyl chloride (prepared according to the method detailed in J. Het. Chem. 1981, 18(5), 997-1006, at pages 997-998 and Table I, for preparation of compound III(a) from 3,5-dimethyl pyrazole) (106 mg, 0.55 mmol) and triethylamine (0.15 mL, 1.1 mmol) in dichloromethane (10 mL) was stirred at reflux for 18 hours. Additional sulphonyl chloride (70 mg, 0.36 mmol) and triethylamine (1 mL) were added, and the reaction stirred at reflux for a further 24 hours. The cooled mixture was washed with 10% citric acid solution, the layers separated, and the organic phase evaporated under reduced pressure. The crude product was purified by column chromatography on silica gel using dichloromethane:methanol (98:2) as eluant and the product recrystallised from ethyl acetate to afford the title compound as a white crystalline solid, 121 mg.

¹H NMR (DMSO-d₆, 400 MHz) δ: 1.42-1.69 (m, 8H), 2.18-2.36 (m, 6H), 2.44 (s, 3H), 2.97 (m, 1H), 3.78 (m, 1H), 6.93 (d, 1H), 7.09 (m, 2H), 7.22 (d, 1H), 7.34 (t, 1H), 7.98 (m, 1H), 8.22 (m, 2H), 12.79 (brs, 1H).

LRMS: m/z ES⁺ 556 [MNa]⁺

Microanalysis found; C, 53.91; H, 5.66; N, 12.05. C₂₄H₂₈FN₅O₄S₂ requires C, 54.02; H, 5.29; N, 12.34%.

### EXAMPLE 11

### Syn-5-Fluoro-2-(3-methylsulphanyl-phenoxy)-N-[4-(quinoline-8-sulphonylamino)-cyclohexyl]-nicotinamide

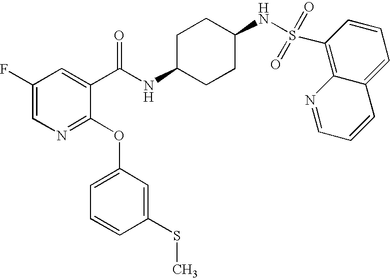

The title compound was obtained as a white crystalline solid after crystallisation from isopropyl acetate, from the amine from preparation 13 and quinoline-8-sulphonyl chloride following a similar procedure to that described in example 10.

¹H NMR (DMSO-d₆, 400 MHz) δ: 1.26-1.65 (m, 8H), 2.51 (s, 3H), 3.22 (m, 1H), 3.74 (m, 1H), 6.92 (m, 2H), 7.07 (s, 1H), 7.13 (d, 1H), 7.37 (t, 1H), 7.64 (m, 1H), 7.75 (t, 1H), 7.96 (m, 1H), 8.19 (d, 1H), 8.23 (s, 1H), 8.29 (m, 2H), 8.54 (d, 1H), 8.97 (d, 1H).

LRMS: m/z ES⁺ 589 [MNa]⁺

Microanalysis found; C, 59.08; H, 4.81; N, 9.78. C₂₈H₂₇FN₄O₄S₂ requires C, 59.35; H, 4.80; N, 9.89%.

### EXAMPLE 12

### Syn-N-[4-(Benzo[1,2,5]oxadiazole-4-sulphonylamino)-cyclohexyl]-5-fluoro-2-(3-methylsulphanyl-phenoxy)-nicotinamide

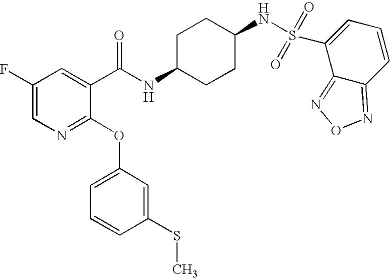

The title compound was obtained after crystallisation from isopropyl acetate, from the amine from preparation 13 and benzofurazan-4-sulphonyl chloride, following a similar procedure to that described in example 10.

¹H NMR (DMSO-d₆, 400 MHz) δ: 1.42-1.70 (m, 8H), 2.45 (s, 3H), 3.40 (m, 1H), 3.79 (m, 1H), 6.92 (d, 1H), 7.05 (s, 1H), 7.11 (d, 1H), 7.34 (t, 1H), 7.81 (m, 1H), 7.96 (m, 1H), 8.05 (d, 1H), 8.20 (m, 3H), 8.36 (d, 1H).

LRMS: m/z ES⁺ 580 [MNa]⁺

Microanalysis found; C, 53.74; H, 4.40; N, 12.36. C₂₅H₂₄FN₅O₅S₂ requires C, 53.85; H, 4.34; N, 12.56%.

### EXAMPLE 13

### Syn-N-[4-(Benzo[1,2,5]thiadiazole-4-sulphonylamino)-cyclohexyl]-5-fluoro-2-(3-methylsulphanyl-phenoxy)-nicotinamide

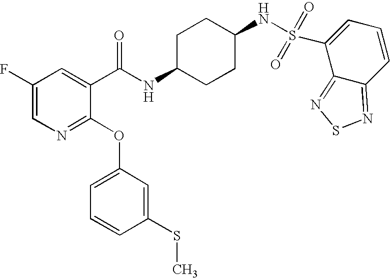

A mixture of the amine from preparation 13 (150 mg, 0.36 mmol), benzo-2,1,3-thiadiazole-4-sulphonyl chloride (128 mg, 0.55 mmol) and triethylamine (0.15 mL, 1.1 mmol) in dichloromethane (10 mL) was stirred at reflux for 18 hours. The cooled mixture was washed with 10% citric acid solution, the layers separated, and the organic phase evaporated under reduced pressure. The crude product was purified by column chromatography on silica gel using dichloromethane:methanol (98:2) as eluant and the product recrystallised from ethyl acetate to afford the title compound as a yellow crystalline solid, 194 mg.

¹H NMR (DMSO-d₆, 400 MHz) δ: 1.38-1.64 (m, 8H), 2.45 (s, 3H), 3.43 (m, 1H), 3.76 (m, 1H), 6.92 (m, 1H), 7.08 (s, 1H), 7.11 (d, 1H), 7.35 (t, 1H), 7.81 (m, 2H), 7.96 (m, 1H), 8.20 (m, 3H), 8.37 (d, 1H).

LRMS: m/z ES⁺ 596 [MNa]⁺

Microanalysis found; C, 52.23; H, 4.30; N, 11.87. C₂₅H₂₄FN₅O₄S₃ requires C, 52.34; H, 4.22; N, 12.21%.

### EXAMPLE 14

### Syn-N-[5-Hydroxy-4-(benzo[1,2,5]thiadiazole-4-sulphonylamino)-cvclohexyl]-5-fluoro-2-(3-methylsulphanyl-phenoxy)-nicotinamide

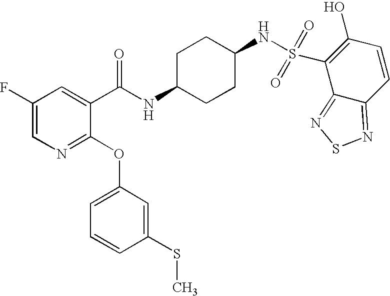

A solution of the ether from preparation 16 (94 mg, 0.15 mmol) in hydrobromic acid in acetic acid (30%, 2 mL) was stirred at 100° C. for 7 hours. The cooled reaction was partitioned between water and dichloromethane, the layers separated, and the organic layer evaporated under reduced pressure. The residual oil was purified by HPLC using a Luna C8(II), 10 μm column and acetonitrile: 0.1% aqueous trifluoroacetic acid (50:50 to 100:0) as eluant. The product was crystallised from dichloromethane:diisopropyl ether to give the title compound as a yellow solid, 26 mg.

¹H NMR (DMSO-d₆, 400 MHz) δ: 1.34-1.69 (m, 8H), 2.45 (s, 3H), 3.31 (m, 1H), 3.76 (m, 1H), 6.92 (d, 1H), 7.05 (s, 1H), 7.11 (d, 1H), 7.35 (t, 1H), 7.47 (d, 1H), 7.78 (m,1H), 7.96 (d,1H), 8.20 (m, 3H), 11.09 (brs,1H).

HRMS: m/z ES⁺ 590.0995 [C₂₅H₂₄FN₅O₅S₃+H]⁺ req 590.0996

### EXAMPLE 15

### Syn-5-Fluoro-N-[4-(2-hydroxy-4-methyl-benzenesulphonylamino)-cyclohexyl]-2-(3-methylsulphanyl-phenoxy)-nicotinamide

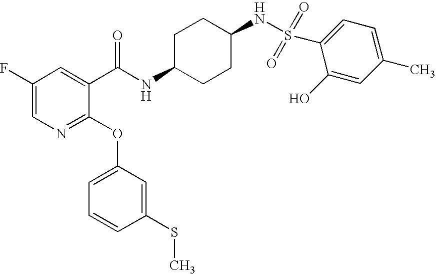

Boron tribromide (1.29 mL, 1M in dichloromethane, 1.29 mmol) was added to an ice-cooled solution of the ether from preparation 18 (180 mg, 0.32 mmol) in dichloromethane (10 mL), and the reaction stirred for 2 hours. Sodium carbonate solution (10 mL) was added, and the mixture stirred at room temperature for 1 hour, until all the solids had dissolved. The mixture was acidified using 2M hydrochloric acid, and the layers separated by filtration through a filter cartridge. The organic solution was concentrated under reduced pressure and the residue purified by column chromatography on silica gel using dichloromethane:methanol (98:2). The product was triturated with diisopropylether to afford the title compound as a white foam, 127 mg.

¹H NMR (DMSO-d₆, 400 MHz) δ: 1.43-1.71 (m, 8H), 2.24 (s, 3H), 2.44 (s, 3H), 3.01 (m, 1H), 3.77 (m, 1H), 6.68-6.78 (m, 3H), 6.92 (m, 1H), 7.08 (m, 2H), 7.35 (t, 1H), 7.49 (d, 1H), 7.98 (m, 1H), 8.21 (m, 2H), 10.44 (s, 1H).

LRMS: m/z ES⁺ 568 [MNa]⁺

Microanalysis found; C, 56.76; H, 5.19; N, 7.68. C₂₆H₂₈FN₃O₅S₂; 0.1 H₂O requires C, 57.04; H, 5.19; N, 7.68%.

### EXAMPLE 16

### Syn-5-Fluoro-N-[4-(2-hydroxy-5-methyl-benzenesulphonylamino)-cyclohexyl]-2-(4-fluoro-3-methylsulphanyl-Phenoxy)-nicotinamide

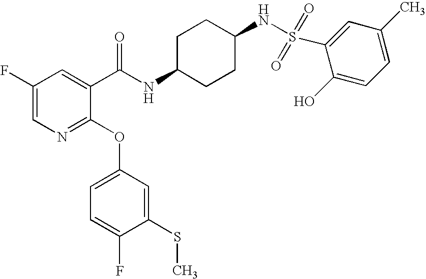

The title compound was obtained as an off-white solid after crystallisation from isopropyl acetate, from the ether from preparation 17, following a similar procedure to that described in example 15 wherein the product was purified by crystallisation from isopropyl acetate, rather than via purification by chromatography.

¹H NMR (DMSO-d₆, 400 MHz) δ: 1.42-1.72 (m, 8H), 2.21 (s, 3H), 2.43 (s, 3H), 3.03 (m, 1H), 3.78 (m, 1H), 6.73 (d, 1H), 6.86 (d, 1H), 7.01 (m, 1H), 7.15-7.32 (m, 3H), 7.42 (s, 1H), 7.98 (m, 1H), 8.21 (m, 2H), 10.32 (s, 1H).

LRMS: m/z ES⁺ 586 [MNa]⁺

Microanalysis found; C, 55.67; H, 5.04; N, 7.06. C₂₆H₂₇F₂N₃O₅S₂ requires C, 55.41; H, 4.83; N, 7.46%.

In Vitro Activity of the Nicotinamide Derivatives

The PDE4 inhibitory activity of the nicotinamide derivatives of the formula (1) is determined by the ability of compounds to inhibit the hydrolysis of cAMP to AMP by PDE4 (see also reference 1). Tritium labelled cAMP is incubated with PDE4. Following incubation, the radiolabelled AMP produced is able to bind yttrium silicate SPA beads. These SPA beads subsequently produce light that can be quantified by scintillation counting. The addition of a PDE4 inhibitor prevents the formation of AMP from cAMP and counts are diminished. The IC₅₀ of a PDE4 inhibitor can be defined as the concentration of a compound that leads to a 50% reduction in counts compared to the PDE4 only (no inhibitor) control wells.

The anti-inflammatory properties of the nicotinamide derivatives of the formula (1) are demonstrated by their ability to inhibit TNFα release from human peripheral blood mononuclear cells (see also reference 2). Venous blood is collected from healthy volunteers and the mononuclear cells purified by centrifugation through Histopaque (Ficoll) cushions. TNFα production from these cells is stimulated by addition of lipopolysaccharide. After 18 hours incubation in the presence of LPS, the cell supernatant is removed and the concentration of TNFα in the supernatant determined by ELISA. Addition of PDE4 inhibitors reduces the amount of TNFα produced. An IC₅₀ is determined which is equal to the concentration of compound that gives 50% inhibition of TNFα production as compared to the LPS stimulated control wells.

All the examples were tested in the assay described above and found to have an IC₅₀ (TNFα screen) of less than 20 nM. And for most of the tested compounds, they were found to have an IC₅₀ (TNFα screen) of even less than 10 nM.

For illustrating purpose, the following table indicates the exact IC₅₀ (TNFα screen) of some representative examples of the present invention which have an IC₅₀ (TNFα screen) of even less than 5 nM:

| Example N^{o} | IC₅₀ (nM) |
|---|---|
| 11 | 0.07 |
| 12 | 0.3 |
| 13 | 0.09 |
| 14 | 1.0 |
| 15 | 2.0 |
| 16 | 3.5 |

References
1. Thompson J W, Teraski W L, Epstein P M, Strada S J., “Assay of nucleotidephosphodiesterase and resolution of multiple molecular forms of the isoenzyme”, Advances in cyclic nucleotides research, edited by Brooker G, Greengard P, Robinson G A. Raven Press, New York 1979, 10, p. 69-92.
2. Yoshimura T, Kurita C, Nagao T, Usami E, Nakao T, Watanabe S, Kobayashi J, Yamazaki F, Tanaka H, Nagai H., “Effects of cAMP-phosphodiesterase isozyme inhibitor on cytokine production by lipopolysaccharide-stimulated human peripheral blood mononuclear cells”, Gen. Pharmacol., 1997, 29(4), p. 63

1. A compound of formula (I): 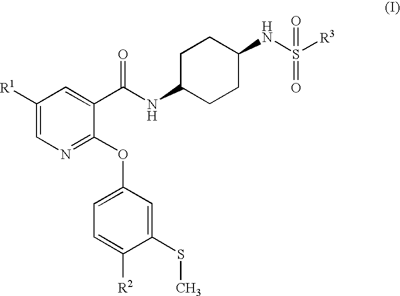
or a pharmaceutically acceptable salt or solvate thereof, wherein:
R¹ and R² are each independently hydrogen, halo or (C₁-C₃)alkyl;
R³ is a 9- or 10-membered bicyclic heteroaryl incorporating one to three heteroatoms independently selected from nitrogen, oxygen and sulphur; phenyl; or a 5- or 6-membered heteroaryl containing from 1 to 3 nitrogen atoms;
said heteroaryl and said phenyl in the definition of R³ are each optionally substituted independently by one to three hydroxy; cyano; halo; (C₁-C₄)alkyl optionally substituted by 1 to 3 halo; hydroxy(C₁-C₄)alkyl; (C₁-C₄)alkoxy or hydroxy(C₂-C₄)alkoxy.

2. A compound of claim 1, or a pharmaceutically acceptable salt thereof, wherein R¹ is H, F, Cl or methyl.

3. A compound of claim 2, or a pharmaceutically acceptable salt thereof, wherein R¹ is F.

4. A compound of claim 3, or a pharmaceutically acceptable salt thereof, wherein R² is H or F.

5. A compound of claim 4, or a pharmaceutically acceptable salt thereof, wherein R² is H.

6. A compound of claim 1, or a pharmaceutically acceptable salt thereof, wherein R³ is a C-linked 9- or 10-membered bicyclic heteroaryl incorporating one to three heteroatoms selected from nitrogen, oxygen and sulphur.

7. A compound of claim 6, or a pharmaceutically acceptable salt thereof, wherein R³ is indolyl, isoindolyl, indolizinyl, indazolyl, benzoimidazolyl, imidazopyridyl pyrrolopyridazinyl, pyrrolopyridyl, benzotriazolyl, pyrazolopyridyl, imidazopyridyl, quinolyl, isoquinolyl, cinnolinyl, quinoxalinyl, quinazolinyl, phthalazinyl, naphthyridinyl, benzofuranyl, benzothiophenyl, benzoxazolyl, benzothiazolyl, benzothiadiazolyl and benzoxadiazolyl, each being optionally substituted independently with one to three hydroxy; cyano; halo; (C₁-C₄)alkyl optionally substituted by 1 to 3 halo; hydroxy(C₁-C₄)alkyl; (C₁-C₄)alkoxy or hydroxy(C₂-C₄)alkoxy.

8. A compound of claim 6, or a pharmaceutically acceptable salt thereof, wherein R³ is quinolyl, 2,1,3-benzothiadiazolyl and 2,1,3-benzoxadiazolyl, hydroxy; cyano; halo; (C₁-C₄)alkyl optionally substituted by 1 to 3 halo; hydroxy(C₁-C₄)alkyl; (C₁-C₄)alkoxy or hydroxy(C2-C₄)alkoxy.

9. A compound of claim 1, or a pharmaceutically acceptable salt thereof, wherein the bicyclic heteroaryl in the definition of R³ is optionally substituted independently by one to three hydroxy, methyl, ethyl, hydroxymethyl, hydroxyethyl, hydroxyethoxy, F or Cl.

10. A compound of claim 9, or a pharmaceutically acceptable salt thereof, wherein the bicyclic heteroaryl in the definition of R³ is optionally substituted by hydroxy.

11. A compound of claim 1, or a pharmaceutically acceptable salt thereof, wherein R³ is phenyl optionally substituted independently by one to three hydroxy, methyl, ethyl, hydroxymethyl, hydroxyethyl, hydroxyethoxy, F or Cl.

12. A compound of claim 11, or a pharmaceutically acceptable salt thereof, wherein R³ is phenyl optionally substituted independently by one to three hydroxy, methyl, F, CN or CF₃.

13. A compound of claim 1, or a pharmaceutically acceptable salt thereof, wherein R³ is an optionally substituted C-linked 5-membered heteroaryl containing 2 or 3 nitrogen atoms, hydroxy; cyano; halo; (C₁-C₄)alkyl optionally substituted by 1 to 3 halo; hydroxy(C₁-C₄)alkyl; (C₁-C₄)alkoxy and hydroxy(C₂-C₄)alkoxy.

14. A compound of claim 13, or a pharmaceutically acceptable salt thereof, wherein R³ is pyrazole or imidazole, hydroxy; cyano; halo; (C₁-C₄)alkyl optionally substituted by 1 to 3 halo; hydroxy(C₁-C₄)alkyl; (C₁-C₄)alkoxy and hydroxy(C₂-C₄)alkoxy.

15. A compound of 14, or a pharmaceutically acceptable salt thereof, wherein R³ is a C-linked 5-membered heteroaryl containing 2 nitrogen atoms optionally substituted independently by one to three (C₁-C₃)alkyl or (C₁-C₃)alkoxy.

16. A compound of claim 15, or a pharmaceutically acceptable salt thereof, wherein R³ is a C-linked 5-membered heteroaryl containing 2 nitrogen atoms optionally substituted by one to three methyl.

17. Syn-N-(4-Benzenesulfonylamino-cyclohexyl)-5-fluoro-2-(3-methylsulfanyl-phenoxy)-nicotinamide;
Syn-5-Fluoro-N-[4-(3-fluoro-benzenesulfonylamino)-cyclohexyl]-2-(3-methylsulfanyl-phenoxy)-nicotinamide;
Syn-5-Fluoro-N-[4-(4-fluoro-benzenesulfonylamino)-cyclohexyl]-2-(3-methylsulfanyl-phenoxy)-nicotinamide;
Syn-5-Fluoro-2-(3-methylsulfanyl-phenoxy)-N-[4-(toluene-3-sulfonylamino)-cyclohexyl]-nicotinamide;
Syn-5-Fluoro-2-(4-methylsulfanyl-phenoxy)-N-[4-(toluene-3-sulfonylamino)-cyclohexyl]-nicotinamide;
Syn-N-[4-(3-Cyano-benzenesulfonylamino)-cyclohexyl]-5-fluoro-2-(3-methylsulfanyl-phenoxy)-nicotinamide;
Syn-5-Fluoro-2-(3-methylsulfanyl-phenoxy)-N-[4-(2-trifluoromethyl-benzenesulfonylamino)-cyclohexyl]-nicotinamide;
Syn-5-Fluoro-N-[4-(1-methyl-1H-imidazole-4-sulfonylamino)-cyclohexyl]-2-(3-methylsulfanyl-phenoxy)-nicotinamide;
Syn-N-[4-(3,5-Dimethyl-1H-pyrazole-4-sulfonylamino)-cyclohexyl]-5-fluoro-2-(3-methylsulfanyl-phenoxy)-nicotinamide;
Syn-5-Fluoro-2-(3-methylsulfanyl-phenoxy)-N-[4-(quinoline-8-sulfonylamino)-cyclohexyl]-nicotinamide;
Syn-N-[4-(Benzo[1,2,5]oxadiazole-4-sulfonylamino)-cyclohexyl]-5-fluoro-2-(3-methylsulfanyl-phenoxy)-nicotinamide;
Syn-N-[4-(Benzo[1,2,5]thiadiazole-4-sulfonylamino)-cyclohexyl]-5-fluoro-2-(3-methylsulfanyl-phenoxy)-nicotinamide;
Syn-N-[5-Hydroxy-4-(benzo[1,2,5]thiadiazole-4-sulfonylamino)-cyclohexyl]-5-fluoro-2-(3-methylsulfanyl-phenoxy)-nicotinamide;
Syn-5-Fluoro-N-[4-(2-hydroxy-4-methyl-benzenesulfonylamino)-cyclohexyl]-2-(3-methylsulfanyl-phenoxy)-nicotinamide; or
Syn-5-Fluoro-N-[4-(2-hydroxy-5-methyl-benzenesulfonylamino)-cyclohexyl]-2-(4-fluoro-3-methylsulfanyl-phenoxy)-nicotinamide;
or a pharmaceutically acceptable salt or solvate thereof.

18. Syn-5-Fluoro-2-(3-methylsulfanyl-phenoxy)-N-[4-(quinoline-8-sulfonylamino)-cyclohexyl]-nicotinamide;
Syn-N-[4-(Benzo[1,2,5]thiadiazole-4-sulfonylamino)-cyclohexyl]-5-fluoro-2-(3-methylsulfanyl-phenoxy)-nicotinamide; or
Syn-5-Fluoro-N-[4-(2-hydroxy4-methyl-benzenesulfonylamino)-cyclohexyl]-2-(3-methylsulfanyl-phenoxy)-nicotinamide;
or a pharmaceutically acceptable salt or solvate thereof.

19. A pharmaceutical composition comprising a compound of claim 1 or a pharmaceutically acceptable salt or solvate thereof and a pharmaceutically acceptable excipient, diluent or carrier.

20. A method of treating a disease, disorder or condition in which PDE4 inhibition is beneficial in a mammal suffering from a disease, disorder or condition in which PDE4 inhibition is beneficial, said method comprising administering to said mammal in need of such treatment a therapeutically effective amount of a compound of claim 1, a pharmaceutically acceptable salt thereof or a pharmaceutical composition comprising a compound of claim 1 or a pharmaceutically acceptable salt thereof and a pharmaceutically acceptable carrier, diluent or excipient.

21. A method of claim 20 wherein the disease, disorder or condition is selected from:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy
pneumoconiosis of whatever type, etiology, or pathogenesis, in particular pneumoconiosis that is a member selected from the group consisting of aluminosis or bauxite workers' disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,
seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,
rheumatoid arthritis of whatever type, etiology, or pathogenesis, in particular rheumatoid arthritis that is a member selected from the group consisting of acute arthritis, acute gouty arthritis, chronic inflammatory arthritis, degenerative arthritis, infectious arthritis, Lyme arthritis, proliferative arthritis, psoriatic arthritis and vertebral arthritis,
gout, and fever and pain associated with inflammation,
an eosinophil-related disorder of whatever type, etiology, or pathogenesis, in particular an eosinophil-related disorder that is a member selected from the group consisting of eosinophilia, pulmonary infiltration eosinophilia, Loffler's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrotizing vasculitis, atopic dermatitis, allergic dermatitis, contact dermatitis, or allergic or atopic eczema,
urticaria of whatever type, etiology, or pathogenesis, in particular urticaria that is a member selected from the group consisting of immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria and papular urticaria,
conjunctivitis of whatever type, etiology, or pathogenesis, in particular conjunctivitis that is a member selected from the group consisting of actinic conjunctivitis, acute catarrhal conjunctivitis, acute contagious conjunctivitis, allergic conjunctivitis, atopic conjunctivitis, chronic catarrhal conjunctivitis, purulent conjunctivitis and vernal conjunctivitis,
uveitis of whatever type, etiology, or pathogenesis, in particular uveitis that is a member selected from the group consisting of inflammation of all or part of the uvea, anterior uveitis, iritis, cyclitis, iridocyclitis, granulomatous uveitis, nongranulomatous uveitis, phacoantigenic uveitis, posterior uveitis, choroiditis; and chorioretinitis,
psoriasis;
multiple sclerosis of whatever type, etiology, or pathogenesis, in particular multiple sclerosis that is a member selected from the group consisting of primary progressive multiple sclerosis and relapsing remitting multiple sclerosis,
autoimmune/inflammatory diseases of whatever type, etiology, or pathogenesis, in particular an autoimmune/inflammatory disease that is a member selected from the group consisting of autoimmune hematological disorders, hemolytic anemia, aplastic anemia, pure red cell anemia, idiopathic thrombocytopenic purpura, systemic lupus erythematosus, polychondritis, scleroderma, Wegner's granulomatosis, dermatomyositis, chronic active hepatitis, myasthenia gravis, Stevens-Johnson syndrome, idiopathic sprue, autoimmune inflammatory bowel diseases, ulcerative colitis, endocrin opthamopathy, Grave's disease, sarcoidosis, alveolitis, chronic hypersensitivity pneumonitis, primary biliary cirrhosis, juvenile diabetes or diabetes mellitus type I, keratoconjunctivitis sicca, epidemic keratoconjunctivitis, diffuse interstitial pulmonary fibrosis or interstitial lung fibrosis, idiopathic pulmonary fibrosis, cystic fibrosis, glomerulonephritis with and without nephrotic syndrome, acute glomerulonephritis, idiopathic nephrotic syndrome, minimal change nephropathy, inflammatory/hyperproliferative skin diseases, benign familial pemphigus, pemphigus erythematosus, pemphigus foliaceus, and pemphigus vulgaris,
prevention of allogeneic graft rejection following organ transplantation,
inflammatory bowel disease (IBD) of whatever type, etiology, or pathogenesis, in particular inflammatory bowel disease that is a member selected from the group consisting of collagenous colitis, colitis polyposa, transmural colitis, ulcerative colitis and Crohn's disease (CD),
septic shock of whatever type, etiology, or pathogenesis, in particular septic shock that is a member selected from the group consisting of renal failure, acute renal failure, cachexia, malarial cachexia, hypophysial cachexia, uremic cachexia, cardiac cachexia, cachexia suprarenalis or Addison's disease, cancerous cachexia and cachexia as a consequence of infection by the human immunodeficiency virus (HIV),
liver injury,
pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension,
bone loss diseases, primary osteoporosis and secondary osteoporosis,
central nervous system disorders of whatever type, etiology, or pathogenesis, in particular a central nervous system disorder that is a member selected from the group consisting of depression, Alzheimers disease, Parkinson's disease, learning and memory impairment, tardive dyskinesia, drug dependence, arteriosclerotic dementia and dementias that accompany Huntington's chorea, Wilson's disease, paralysis agitans, and thalamic atrophies,
infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex,
yeast and fungus infections wherein said yeast and fungi are sensitive to upregulation by TNF-α or elicit TNF-α production in their host, e.g., fungal meningitis, particularly when administered in conjunction with other drugs of choice for the treatment of systemic yeast and fungus infections, including but are not limited to, polymixins, e.g. Polymycin B, imidazoles, e.g. clotrimazole, econazole, miconazole, and ketoconazole, triazoles, e.g. fluconazole and itranazole as well as amphotericins, e.g. Amphotericin B and liposomal Amphotericin B,
ischemia-reperfusion injury, ischemic heart disease, autoimmune diabetes, retinal autoimmunity, chronic lymphocytic leukemia, HIV infections, lupus erythematosus, kidney and ureter disease, urogenital and gastrointestinal disorders and prostate diseases,
reduction of scar formation in the human or animal body, such as scar formation in the healing of acute wounds, and
psoriasis, other dermatological and cosmetic uses, including antiphlogistic, skin-softening, skin elasticity and moisture-increasing activities.

22. A method of claim 21 wherein the disease, disorder or condition is chronic obstructive pulmonary disease, asthma or chronic bronchitis.

23. A combination of a compound of claim 1 or a pharmaceutically acceptable salt or solvate thereof with other therapeutic agents selected from
(a) 5-Lipoxygenase (5-LO) inhibitors or 5-lipoxygenase activating protein (FLAP) antagonists,
(b) Leukotriene antagonists (LTRAs) including antagonists of LTB4, LTC4, LTD4, and LTE4,
(c) Histaminic receptor antagonists including H1, H3 and H4 antagonists,
(d) α1- and α2-adrenoceptor agonist vasoconstrictor sympathomimetic agents for decongestant use,
(e) Muscarinic M3 receptor antagonists or anticholinergic agents,
(f) β2-adrenoceptor agonists,
(g) Theophylline,
(h) Sodium cromoglycate,
(i) COX-1 inhibitors (NSAIDs) and COX-2 selective inhibitors,
(j) Oral or inhaled Glucocorticosteroids,
(k) Monoclonal antibodies active against endogenous inflammatory entities,
(l) Anti-tumor necrosis factor (anti-TNF-a) agents,
(m) Adhesion molecule inhibitors including VLA-4 antagonists,
(n) Kinin-B1- and B2-receptor antagonists,
(o) Immunosuppressive agents,
(p) Inhibitors of matrix metalloproteases (MMPs),
(q) Tachykinin NK1, NK2 and NK3 receptor antagonists,
(r) Elastase inhibitors,
(s) Adenosine A2a receptor agonists,
(t) Inhibitors of urokinase,
(u) Compounds that act on dopamine receptors, e.g. D2 agonists,
(V) Modulators of the NFkb pathway, e.g. IKK inhibitors,
(w) Agents that can be classed as mucolytics or anti-tussive,
(x) antibiotics, and
(y) p38 MAP kinase inhibitors.
